(19) United States
(12) Patent Application Publication
(10) Pub. No.: US 2012/0108980 A1
(43) Pub. Date: May 3, 2012

(54) CATHETER WITH SHAPE MEMORY ALLOY ACTUATOR

(75) Inventors: Thomas W. Shilling, Lakewood, CO (US); Thomas L. Tolt, Aurora, CO (US); Clyde G. Oakley, Centennial, CO (US); Richard W. Denny, Castle Rock, CO (US); Dennis R. Dietz, Littleton, CO (US); Michael J. Vonesh, Flagstaff, AZ (US); Craig T. Nordhausen, Parker, CO (US)

(73) Assignee: Gore Enterprise Holdings, Inc., Newark, DE (US)

(21) Appl. No.: 13/280,078

(22) Filed: Oct. 24, 2011

Related U.S. Application Data
(60) Provisional application No. 61/405,784, filed on Oct. 22, 2010.

(51) Int. Cl. A61B 8/12

(52) U.S. Cl. 600/466

(57) ABSTRACT

Actuators employable for oscillating movement of a load. An improved actuator may include at least a first shape memory member that is actuatable to affect at least a portion of the oscillating movement of the load. The actuator may further include a second shape memory member actuatable to affect at least a second portion of the oscillating movement of the load. The utilization of one or more shape memory members facilitates the realization of controllable and reliable oscillating movement of a load in a compact manner. Such actuators may be used in imaging catheters having an ultrasound transducer disposed for oscillating movement to scan across an internal region of interest. Such imaging catheters may be used in generating three dimensional and/or real-time three dimensional (4D) images.

### RELATED APPLICATIONS

This application claims priority under 35 U.S.C. §119 to U.S. Provisional Patent Application No. 61/405,784, filed Oct. 22, 2010, entitled “CATHETER WITH SHAPE MEMORY ALLOY ACTUATOR”, which is hereby incorporated by reference in its entirety.

### FIELD OF THE INVENTION

The present invention relates to actuators employable for oscillating movement of a load, and more particularly, to actuators employing one or more shaped memory members. The invention is particularly apt to imaging catheters having an ultrasound transducer disposed for oscillating movement to scan a volume encompassing an internal anatomical region of interest.

### BACKGROUND OF THE INVENTION

Actuators are employed in a variety of applications for controlled movement of a mechanism, or load. Increasingly, actuator applications have been recognized which have small space, high-reliability and low power requirements that present unique design challenges.

Actuators may employ shape memory materials to produce movement. Shape memory materials are materials that experience dimensional changes under application of an external stimulus such as temperature or magnetic field. There are two types of shape memory materials that can achieve thermally-induced reversible shape changes: 1) Shape memory alloys (SMA) that are metallic alloys that undergo reversible phase changes between two different crystallographic phases upon a change in temperature and 2) Shape memory polymers (SMP) that typically consist of two polymer components and two phases, one with a higher melting temperature than the other. When shape memory polymers are heated above a specific glass transition temperature, one phase is generally in a rubbery phase and can deform easily. When subsequently cooled below this glass transition temperature, the SMP retains its given permanent shape. The distinguishing feature of SMP compared to all other polymers is that this dimensional change is marked by a sharp transition temperature and a rubbery plateau, along with a capacity to enable large strains without producing permanent local material damage.

Examples of significant shape memory alloys (SMA) are Nitinol, an alloy of nickel and titanium, copper-base alloys, and FeMnSiCrNi shape memory stainless steels. These metallic alloys are distinguished in that they may be heated to produce a corresponding martensite-to-austenite crystallographic phase transformation which results in a reduction in the length. Subsequent cooling of the shape memory alloy may result in an austenite-to-martensite phase transformation and the shape remains unchanged, whereby it may be returned to its original length under an applied stress. If the shape memory material is operatively associated with other members, the phase changes may be used to generate forces that may be used to create movement of the other members. Such heating may be created by passing a current through the shape memory material.

Catheters are medical devices that may be inserted into a body vessel, cavity or duct, and manipulated utilizing a portion that extends out of the body. Typically, catheters are relatively thin and flexible to facilitate advancement/retraction along non-linear paths. Catheters may be employed for a wide variety of purposes, including the internal bodily positioning of diagnostic and/or therapeutic devices. For example, catheters may be employed to position internal imaging devices (e.g., ultrasound transducers), to deploy implantable devices (e.g., stents, stent grafts, vena cava filters), and/or to deliver therapy (e.g., ablation catheters, drug delivery).

In this regard, use of ultrasonic imaging techniques to obtain visible images of structures is increasingly common. Broadly stated, an ultrasound transducer, typically comprising a number of individually actuated piezoelectric elements arranged in an array, is provided with suitable drive signals such that a pulse of ultrasonic energy travels into the body of the patient. The ultrasonic energy is reflected at interfaces between structures of varying acoustic impedance. The same or a different transducer detects the receipt of the return energy and provides a corresponding output signal. This signal can be processed in a known manner to yield an image, visible on a display screen, of the interfaces between the structures and hence of the structures themselves.

Intracardiac Echocardiography (ICE) catheters have become the preferred imaging modality for use in structural heart intervention because they provide high resolution 2D ultrasound images of the soft tissue structure of the heart. Additionally, ICE imaging does not contribute ionizing radiation to the procedure. ICE catheters can be used by the interventional cardiologist and staff within the context of their normal procedural flow and without the addition of other hospital staff. Current ICE catheter technology does have limitations though. The conventional ICE catheters are limited to generating only 2D images. Furthermore, the clinician must steer and reposition the catheter in order to capture multiple image planes within the anatomy. The catheter manipulation needed to obtain specific 2D image planes requires that a user spend a significant amount of time becoming facile with the catheter steering mechanisms.

The Philips iE33 echocardiography system running the new 3D transesophageal (TEE) probe (available from Philips Healthcare, Andover, Mass., USA) represents the first commercially-available real-time 3D (four dimensional (4D)) TEE ultrasound imaging device. This system provides the clinician with the 4D imaging capabilities needed for more complex interventions, but there are several significant disadvantages associated with this system. Due to the large size of the TEE probe (50 mm circumference and 16.6 mm width), patients need to be anesthetized or heavily sedated prior to probe introduction (G. Hamilton Baker, MD 4t al., Usefulness of Live Three-Dimensional Transesophageal Echocardiography in a Congenital Heart Disease Center, Am J Cardiol 2009; 103: 1025-1028). This requires that an anesthesiologist be present to induce and monitor the patient on anesthesia. In addition the hemodynamic status of the patient may require monitoring. Furthermore, minor and major complications from TEE probe use do occur including complications ranging from sore throat to esophageal perforation. The complexity of the Phillips TEE system and probe require the participation of additional staff such as an anesthesiologist, echocardiographer and ultrasound technician. This increases procedure time and cost.

Of particular interest are imaging catheter applications for small-scale actuators. The present inventors have realized the need for an imaging platform that is catheter-based and small enough for percutaneous access with three dimensional imaging in real-time (4D) capabilities. Using such a catheter-based imaging system for visualizing the three dimensional (3D) architecture of the heart, for example, on a real-time basis during intervention is highly desirable from a clinical perspective as it would facilitate more complex procedures such as left atrial appendage occlusion, mitral valve repair, and ablation for atrial fibrillation. 3D imaging would also allow the clinician to fully determine the relative position of structures. This capability would be of particular import in cases of structural abnormalities in the heart where typical anatomy is not present. Two dimensional transducer arrays provide a means to generate 3D images, but currently available 2D arrays require a high number of elements in order to provide sufficient aperture size and corresponding image resolution. This high element count results in a 2D transducer that is prohibitive with respect to clinically acceptable catheter profiles.

As internal diagnostic and therapeutic procedures continue to evolve, the desirability of enhanced procedure imaging via compact and maneuverable catheters has been recognized by the present inventors. More particularly, the present inventors have recognized the desirability of providing catheter features that facilitate selective positioning and actuator control of imaging componentry (e.g., to produce real time 3D images) located at a distal end of a catheter, while maintaining a relatively small profile, thereby yielding enhanced functionality for various clinical applications. As may be appreciated, the utilization of ultrasound transducers on catheters presents dimensional challenges, particularly for vascular applications. For example, for cardiovascular applications it may be desirable to maintain a maximum cross-dimension of less than about 12 French (Fr), and more preferably less than about 10 Fr, during advancement of an imaging catheter into the right atrium or other chambers of the heart. Due to the size constraints of some anatomical locations, e.g., that in the heart, it is desirable that the selective positioning necessary to achieve desired viewing angles be obtainable within a small anatomical volume such as, for example, a volume with a maximum cross dimension of less than about 3 cm.

### SUMMARY OF THE INVENTION

The present invention relates to actuators employable for oscillating movement of a load. An improved actuator may include at least a first shape memory member (e.g., comprising a shape memory material) that is actuatable to affect at least a portion of the oscillating movement of the load. In contemplated embodiments, the actuator may further comprise a second shape memory member (e.g., comprising a shape memory material) actuatable to affect at least a second portion of the oscillating movement of the load. The utilization of one or more shape memory members facilitates the realization of controllable and reliable oscillating movement of a load in a compact and low-power manner. The first and second shape memory members may be actuatable in at least partially-offset timed relation to affect at least a portion of the oscillating movement of the load.

In one aspect, the actuator may include an enclosure defining an enclosed volume. The enclosed volume may contain a fluid. The fluid may be a liquid (e.g., to facilitate acoustic signal transmission). At least a portion of a first shape memory member of an actuator may be immersed within the fluid, and a first thermal insulation layer may be disposed about the immersed portion of the first shape memory member. Similarly, at least a portion of a second shape memory member of an actuator may be immersed within the fluid, and a second thermal insulation layer may be disposed about the immersed portion of the second thermal insulation layer. As may be appreciated, the provision of a thermal insulation layer on one or more shape memory member(s) may advantageously affect the rate of transfer of thermal energy between the contained fluid and the shape memory member(s). In such an aspect, for example, the load may comprise an ultrasound transducer.

In an implementation, the load is immersed within the fluid and disposed for oscillating movement through an angular range about a pivot axis within the enclosed volume, wherein the pivot axis is fixed relative to the enclosed volume. In this regard, the actuator may include first and second shape memory members operatively associated with the load, wherein the first and second shape memory members are actuatable in at least partially-offset timed relation to affect at least a portion of the pivotal movement of the load. Such an implementation, for example, may be in the form of a catheter having an elongate catheter body and a distal end portion supportably disposed at the distal end of the catheter body and defining the enclosed volume containing the load and the fluid. In such an implementation, the load may be an ultrasound transducer and the ultrasound transducer may be immersed in the fluid for ultrasound signal transmission and/or receipt.

In certain embodiments, the first and second shape memory members may be interconnected to the load within the enclosed volume and immersed within the contained fluid. In turn, first and second thermal insulation layers may be disposed about at least a portion of the first and second shape memory members, respectively, within the enclosed volume and immersed within the fluid. Further, the first and second shape memory members may be individually insulated for electrical isolation.

In arrangements, the first and/or second thermal insulation layers may have a thermal conductance of between about 0.03 watts per meter per Kelvin (W/mK) and 0.20 W/mK when measured at about 25° C. In arrangements, the first and/or second thermal insulation layers may have a thermal conductance of between about 0.05 W/mK and 0.08 W/mK when measured at about 25° C. In one approach, the first and/or second thermal insulation layers may comprise a fluoropolymer. In one implementation, the first and/or second thermal insulation layers may comprise at least one material selected from a group consisting of: a polytetrafluoroethylene (PTFE), an expanded polytetrafluoroethylene (ePTFE), an electrostatic spray-coated PTFE, a fluorinated ethylene propylene, an expanded fluorinated ethylene propylene, a perfluoroalkoxy copolymer, a polyvinylidene fluoride, a polyurethane, a silicone rubber, a plasma-coated polymer film (e.g., a low temperature plasma-enhance trimethylsilane), PARYLENE™, and blends and copolymers thereof. Other materials having a similar thermal conductance may also be employed. In one approach, the first and/or second thermal insulation layers may comprise a microporous material.

In addition to first and/or second thermal insulation layers as noted above, the actuator may include corresponding first and/or second outer layers, respectively, disposed (e.g., adherently disposed) about the first and/or second thermal insulation layers, respectively. In this regard, the first and/or second outer layers may be advantageously adapted for immersion within the contained fluid within the enclosure. In this regard, the first and/or second outer layers may each comprise a hydrophobic material. In one approach, the first and/or second outer layers may be selected to have a surface energy of less than about 50 dyn/cm². Additionally, or alternatively, the first and/or second outer layers may be selected to have a dielectric withstand voltage of at least about 500 kV/m.

In an approach, in addition to the thermal properties of the first and/or second thermal insulation layers as noted above, the first and/or second thermal insulation layers may be advantageously adapted or configured for immersion within the contained fluid within the enclosure. In this regard, first and/or second thermal insulation layers may perform both the above-described function of the first and/or second thermal insulation layers and the above-described function of the first and/or second outer layers. Thus, the first and/or second thermal insulation layers may each comprise a hydrophobic material. In one approach, the first and/or second thermal insulation layers may be selected to have a surface energy of less than about 50 dyn/cm². Additionally, or alternatively, the first and/or second thermal insulation layers may be selected to have a dielectric withstand voltage of at least about 500 kV/m. In this regard, the first and/or second thermal insulation layers may be capable of providing the above-noted insulative properties along with the above-noted hydrophobicity and dielectric withstand voltage.

Layers disposed about at least a portion of the first and second shape memory members, such as the above-described first and/or second thermal insulation layers and the above-described first and/or second outer layers, may have an elongation modulus that allows the layers to move with the shape memory members as the shape memory members change length. In this regard, the layers may be operable to elongate and shrink along with the shape memory members without peeling, cracking or delaminating. The layers may be adhesively joined to the shape memory members.

In an embodiment, within the enclosed volume, electrically active components may be insulated to limit undesired current flow (e.g., short circuiting). Such electrically active components may include, for example, electrical interconnections to the shape memory members and ultrasound transducer immersed in the fluid. Such insulation may be particularly beneficial where the fluid within the enclosed volume is a liquid.

In another aspect, a first shape memory member may be actuatable to rotate a load (e.g., an ultrasound transducer) in a first direction about the pivot axis. Conversely, a second shape memory member may be actuatable to rotate the load (e.g., an ultrasound transducer) in a second direction about the pivot axis, wherein the first direction is opposite to the second direction.

In an arrangement, the shape memory members may be operable to vary in length by at least about 1% due to actuation (e.g., by heating by passing current therethrough). In another arrangement, the shape memory members may be operable to vary in length by at least about 2% due to actuation. In a particular arrangement, the shape memory members may be varied in length by about 4% due to actuation.

In various embodiments, the first and second shape memory members may be defined by corresponding first and second shape memory wire lengths, respectively. In one approach, the first and second shape memory wire lengths may comprise physically-separate first and second wires. In another approach, the first and second shape memory wire lengths may be defined by different segments, e.g., first and second lengths, respectively, of a continuous shape memory wire.

A first end of the first shape memory wire length may be interconnected in fixed relation to one of an enclosure (e.g., at a distal end portion of a catheter) and a load (e.g., an ultrasound transducer) on a first side of the pivot axis. Similarly, a first end of the second shape memory wire length may be interconnected in fixed relation to one of the enclosure (e.g., at a distal end portion of a catheter) and a load (e.g., an ultrasound transducer) on a second side of the pivot axis, opposite to the first side.

In one approach, the first shape memory wire length may be interconnected to a corresponding other one of the load (e.g., an ultrasound transducer) and enclosure at a first interconnection location. Further, the second shape memory wire length may be interconnected to a corresponding other one of the load (e.g., an ultrasound transducer) and enclosure at a second interconnection location, wherein the first and second interconnection locations are located on opposite sides of the pivot axis.

In one embodiment, each of the first and second shape memory wire lengths may have corresponding second ends that are interconnected in fixed relation to the corresponding one of the enclosure and the load (e.g., an ultrasound transducer). Further, the first and second shape memory wire lengths may be interconnected between their opposing first and second ends to the corresponding other one of the enclosure and the load (e.g., an ultrasound transducer). In this regard, the noted first and second interconnection locations may be offset on opposite sides of the pivot axis. In one implementation, the first and second offset locations may be substantially equidistance from the pivot axis. In such arrangement, the first and second shape memory wire lengths may be symmetrically disposed relative to the load (e.g., an ultrasound transducer).

The first and second shape memory wire lengths may be disposed to each include corresponding first and second portions thereof that correspondingly define first and second included angles. In turn, the first and second shape memory wire lengths may be arranged so that the first and second included angles increase and decrease to displace the load in response to corresponding actuation and deactuation of the first and second shape memory members, respectively. By arranging the first and second shape memory wire lengths to include such included angles, an effective displacement of at least about 10% to 20% of the wire length may be achieved. Stated differently, an effective elongation of at least about 10% to 20% may be achieved, wherein an effective elongation is the elongation that would be needed to produce a similar movement of a load by a shape memory member disposed generally perpendicular to the load and disposed within a similar volume as the shape memory wire lengths with included angles.

In another embodiment, the first shape memory wire length may comprise a first end interconnected to an enclosure (e.g., at a distal end portion of actuator) on a first side of the pivot axis, and a second end interconnected to the load (e.g., an ultrasound transducer) on a second side of the pivot axis opposite to the first side. Similarly, the second shape memory wire length may have a first end interconnected to the enclosure on the first side of the pivot axis, and a second end interconnected to the load (e.g., an ultrasound transducer) on the second side of the pivot axis.

In yet another embodiment, the first shape memory wire length may comprise first and second ends interconnected in fixed relation to one of an enclosure (e.g., at a distal end portion of a catheter) and a load (e.g., an ultrasound transducer). Further, an engagement member (e.g., a stanchion, post, etc.) may be provided in fixed relation to the other one of the enclosure and the load, wherein the first shape memory wire length engages the engagement member to rotate the load in the first direction during actuation of the first shape memory wire length. Similarly, the second shape memory wire length may comprise a first end and a second end interconnected in fixed relation to said one of the enclosure and the load, wherein the second shape memory wire length engages the engagement member to rotate the load in a second direction during actuation of the second shape memory wire length.

In some embodiments, a central axis of a load (e.g., an ultrasound transducer) may be parallel to the pivot axis. In other embodiments, such central axis may be coincide with the pivot axis.

In various embodiments, a drive energy source may be included for repeatedly providing first and second energy signals during corresponding first and second time periods to the first and second shape memory members, respectively. The drive energy source may be operable to define a first time interval between an end of each first time period and a start of each second time period, wherein at least the second shape memory member is provided to be in elastic tension during at least a portion of each first time interval so that the second shape memory member is operable to affect at least a portion of the oscillating, pivotal movement of the load (e.g., an ultrasound transducer) during each first time interval. Further, the drive energy source may be operable to repeatedly provide the first and second energy signals with a second time interval defined between an end of each second time period and the start of each first time period. In turn, the first shape memory member may be provided to be in elastic tension during at least a portion of each second time interval so that the first shape memory member is operable to affect at least a portion of the oscillating, pivotal movement of the load (e.g., an ultrasound transducer) during each second time interval. As may be appreciated, the first and second shape memory members may be utilized to affect different portions of the oscillating, pivotal movement of the load corresponding with opposite end portions of an angular range of the pivotal movement.

In certain implementations, at least a first magnetic member may be supportably interconnected to one of an enclosure (e.g., at a distal end portion of a catheter) and a load (e.g., an ultrasound transducer), and located to affect at least a portion of the oscillating, pivotal movement of the load (e.g., an ultrasound transducer). In one approach, the first magnetic member may include a permanent magnet; for example, a permanent magnet comprising coated neodymium iron boron or samarium cobalt. In another approach, the first magnetic member may comprise an electromagnetic member.

Relatedly, a second magnetic member may be supportably interconnected to one of the enclosure and the load to affect at least a second portion of the oscillating, pivotal movement of the load. In this regard, the first and second portions of the oscillating, pivotal movement of the load may correspond with opposite end portions of a predetermined angular range of pivotal movement of the load. In certain implementations, the first magnetic member and/or second magnetic member may be operable to apply an attractive force. Similarly, in certain arrangements the first magnetic member and/or second magnetic member may be operable to apply a repulsive force. The application of force by the first and/or second magnetic members may be to a magnetizable member interconnected to the other one of the enclosure and the load. In another implementation, the application of force by the first and/or second magnetic members may be to at least one additional magnetic member interconnected to the other one of the enclosure and the load.

As noted, the above-described actuators are particularly apt for catheter implementations. In this regard, the first and second shape memory members may be disposed in an enclosure for affecting oscillating movement of an ultrasound transducer array at a distal end portion of the catheter. Further, the distal end portion may be provided to be selectively positionable by a user relative to a catheter body. In some embodiments, the distal end portion may be provided to be selectively angled across a range of angles relative to a catheter body. By way of example, the catheter may include a hinge for interconnecting the distal end portion to the catheter body. In other embodiments, the distal end portion may be provided to be selectively rotated about a range of angles relative to a catheter body.

In still another aspect, a method of affecting oscillating, pivoting motion of a load is provided. The method may include first actuating a first shape memory member operatively associated with the load to pivot the load in a first direction, and then second actuating a second shape memory member operatively associated with the load to pivot the load in a second direction opposite to the first direction. The method may further include repeating the first and second actuating steps in accordance with a predetermined cycle to affect oscillating, pivotal movement of the load through an angular range relative to a pivot axis. In an embodiment, the method may be a method for use in a catheter where the load is an ultrasound transducer immersed within a fluid and disposed for pivotal movement about the pivot axis within the enclosed volume where the enclosed volume is defined by a distal end portion supportably disposed at a distal end of an elongate catheter body. In such an embodiment, the method may further include operating the ultrasound transducer to transmit and/or receive acoustic signals through the fluid during at least a portion of each occurrence of the first and/or second actuating steps.

In an approach, the first actuating step may include first applying a first electrical signal to the first shape memory member to change the first shape memory member from a first configuration to a second configuration and thereby impart a first force to the load. The approach may also include the second actuating step comprising second applying a second electrical signal to the second shape memory member to change the second shape memory member from a first configuration to a second configuration and thereby impart a second force to the load. The method may include using the first force to return the second shape memory member from its second configuration to its first configuration, and using the second force to return the first shape memory member from its second configuration to its first configuration.

In an implementation, the oscillating, pivotal movement of the ultrasound transducer achieved by repeating the first and second actuating steps may occur at a rate between 1 and 50 Hz, or between 8 and 30 Hz. In another implementation, the oscillating, pivotal movement of the ultrasound transducer achieved by repeating the first and second actuating steps may occur at a rate of at least 10 Hz; in still another implementation, the rate may be at least 50 Hz.

In an arrangement, the first shape memory member may shorten during the first applying step, and the second shape memory member may shorten during the second applying step. The shape memory members may be in the form of shape memory wires.

In various embodiments, the first and second shape memory members may be defined by corresponding first and second shape memory wire lengths, respectively. In one approach, the first and second shape memory wire lengths may comprise physically-separate first and second wires. In another approach, the first and second shape memory wire lengths may be defined by different first and second lengths, respectively, of a continuous shape memory wire. The first and second portions may be defined by different first and second lengths, respectively, of a continuous shape memory wire, or by physically-separate first and second wires.

In certain implementations, the first and second shape memory members may each include corresponding first and second portions that define corresponding first and second included angles, respectively. In such implementations, the method may include increasing the first included angle and decreasing the second included angle during the first applying step, and increasing the second included angle and decreasing the first included angle during the second applying step.

In an approach, the predetermined cycle may include a first time interval between an end of the first applying step and a start of the second applying step. Such an approach may include employing an elastic response of the second shape memory member during each first interval to initiate pivotal movement of the load in the second direction. The predetermined cycle may include a second time interval between an end of the second applying step and a start of the first applying step, and the present approach may further include employing an elastic response of the first shape memory member during each occurrence of the second interval to initiate pivotal movement of the load in the first direction.

In an arrangement, the method may include employing a magnet to apply a magnetic force to the load to affect at least a portion of the oscillating pivotal movement. The method may also include employing a second magnet to apply a magnetic force to affect at least a different portion of the oscillating pivotal movement. In one approach, the first and second magnets may affect opposite end portions of the angular range.

Numerous additional features and advantages of the present invention will become apparent to those skilled in the art upon consideration of the embodiment descriptions provided hereinbelow.

### BRIEF DESCRIPTION OF THE DRAWINGS

FIG. 1 is a side view of one embodiment of an actuator comprising the present invention.

FIG. 2A is a perspective view of selected components of the actuator embodiment of FIG. 1.

FIG. 2B is a perspective view of selected components of the actuator embodiment of FIG. 1 along with alternative actuator components.

FIGS. 3A and 3B are end views of selected componentry of the actuator embodiment of FIG. 1 shown at different times of operation.

FIG. 3C is an end view of selected componentry of the actuator embodiment of FIG. 1 with a first example of magnetic assist.

FIG. 3D is an end view of selected componentry of the actuator embodiment of FIG. 1 with a second example of magnetic assist.

FIG. 4A is a side view of another embodiment of an actuator comprising the present invention.

FIG. 4B is a side view of an additional embodiment of an actuator comprising the present invention.

FIG. 4C is a side view of a further embodiment of an actuator comprising the present invention.

FIGS. 5A, 5B and 5C are end views of selected componentry of the actuator embodiment of FIG. 4A shown at different times of operation.

FIGS. 5AA, 5BB and 5CC are end views of selected componentry of a modified arrangement of the actuator embodiment off FIG. 4A shown at different times of operation.

FIG. 6 is a side view of another embodiment of an actuator comprising the present invention.

FIG. 7 is a side view of another embodiment of an actuator comprising the present invention.

FIGS. 8 and 9 illustrate a distal end of a catheter body connected by a hinge to the actuator embodiment of FIG. 7.

FIG. 10 illustrates an ultrasound imaging system with a handle, a catheter, and the actuator embodiment of FIG. 7.

FIGS. 11 and 12 show placement of a steerable catheter embodiment that includes the actuator embodiment of FIG. 7 for intracardiac echocardiography within the right atrium of the heart.

FIG. 13 shows placement of the embodiment of FIG. 11 in the right atrium of the heart with the actuator embodiment of FIG. 7 in a second position.

FIG. 14 shows placement of the embodiment of FIG. 11 in the right atrium of the heart with the actuator embodiment of FIG. 7 in a third position.

FIG. 15A is a graph of a drive signal used to drive shape memory members and of the corresponding position of a load being driven.

FIG. 15B is a graph of another drive signal used to drive shape memory members and of the corresponding position of a load being driven.

### DETAILED DESCRIPTION

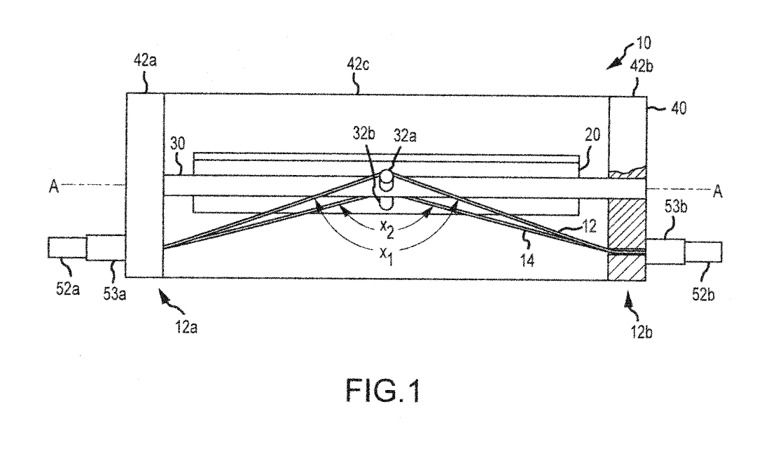

FIG. 1 illustrates one embodiment of an actuator 10 comprising a first shape memory member 12 and a second shape memory member 14 that are actuatable to effect oscillating, pivotal movement of a load 20 about a pivot axis AA. In this regard, pivot axis AA may be defined by a shaft member 30 which is journaled at each end and rotatable relative to an enclosure 40. The enclosure 40 includes a first end piece 42a, a second end piece 42b, and an outer shell 42c (shown as transparent in FIG. 1). In turn, load 20 may be supportably mounted to the shaft member 30 for pivoting movement therewith.

The first and second shape memory members 12, 14 may each comprise a length of shape memory material (e.g., Nitinol, a metal alloy of nickel and titanium), wherein the first and second shape memory members 12, 14 may be heated in at least partially offset timed relation to yield corresponding martensitic-to-austenitic phase transformation and corresponding reductions (e.g., shrinkage) in the length of each member. As will be appreciated, such alternating length reductions causes shaft member 30 to rotate back and forth, thereby causing load 20 to pivot back and forth about pivot axis AA in an oscillating manner. Such heating may be achieved by applying electrical energy to the shape memory members 12, 14. The applied energy may be in the form of an applied voltage that induces a current flow in the shape memory members 12, 14, which produces the heating. The first and second shape memory members 12, 14 may each comprise a length of shape memory wire or any other appropriate shape memory form (e.g., a shape memory ribbon, a multiple element member such as a multiple filament wire, a coil, a helically wound strand).

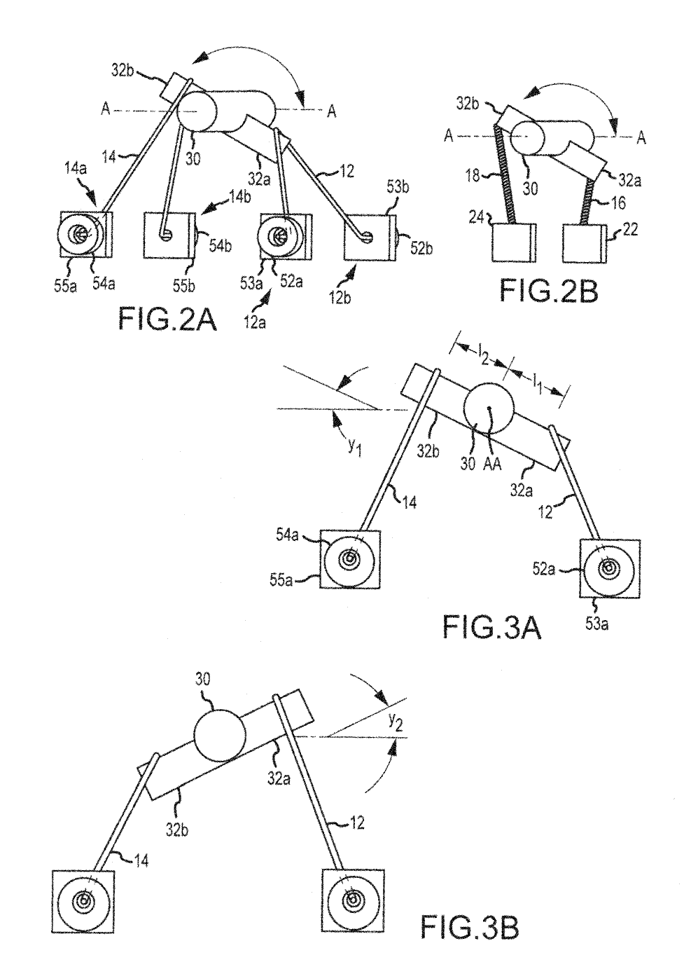

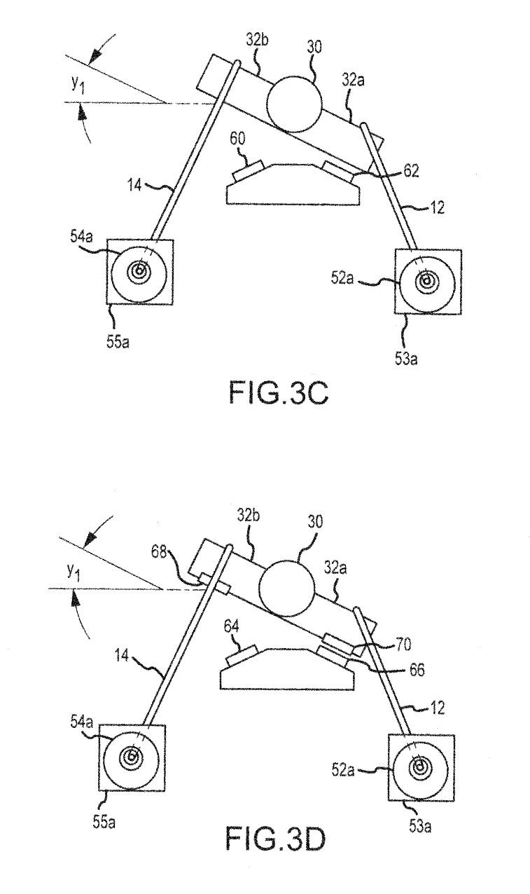

Reference is now made to FIG. 1, together with FIGS. 2A, 3A and 3B which illustrate the operative interface between the first shape memory member 12, the second shape memory member 14 and shaft member 30. For explanatory purposes, the load 20, first and second end pieces 42a, 42b, and the outer shell 42c are not shown in FIGS. 2A through 3D. In the illustrated embodiment, first shape memory member 12 may be fixedly interconnected at a first end 12a to an anchor 52a. The anchor 52a may be interconnected to an elastically deformable member (e.g., a spring-like member such as a resilient, compressible member) 53a, which in turn is interconnected to first end piece 42a. In this regard, via compression of the elastically deformable member 53a, anchor 52a is able to move a limited amount relative to the first end piece 42a. First shape memory member 12 may be fixedly interconnected at a second end 12b to an anchor 52b (partly visible in FIG. 2A). Likewise, the anchor 52b may be interconnected to an elastically deformable member 53b, which in turn is interconnected to second end piece 42b. Similarly, second shape memory member 14 may be fixedly interconnected at a first end 14a to an anchor 54a. The anchor 54a may be interconnected to an elastically deformable member 55a, which in turn is interconnected to first end piece 42a. Second shape memory member 14 may be fixedly interconnected at a second end 14b to an anchor 54b (partly visible in FIG. 2A). The anchor 54b may be interconnected to an elastically deformable member 55b, which in turn is interconnected to second end piece 42b.

The elastically deformable members 53a, 53b, 55a, 55b may be operable to elastically deform (e.g., resiliently compress and uncompress) in a manner that compensates for possible mismatches between the lengths of the shape memory members 12, 14 as they simultaneously change length (e.g., one of the shape memory members 12, 14 may be contracting in length as the other is lengthening). By compressing, the elastically deformable members 53a, 53b, 55a, 55b may help to prevent excessive elastic tension in the shape memory members 12, 14. Additionally, the elastically deformable members 53a, 53b, 55a, 55b may help compensate for elastic tension variations due to changes in geometry as the shape memory members 12, 14 pivot during load 20 oscillating movement.

The first shape memory member 12 may be operatively interconnected to shaft member 30 via engagement member 32a fixedly interconnected to and laterally extending away from shaft member 30 on one side of pivot axis AA. Similarly, second shape memory member 14 may be operatively interconnected to shaft member 30 via engagement member 32b fixedly interconnected to and laterally extending away from shaft member 30 on another side of pivot axis AA. The engagement members 32a, 32b may be grooved to help positively locate the shape memory members 12, 14 relative thereto. In embodiments where the distances between engagement member 32a and anchor 52a, and between engagement member 32a and anchor 52b are unequal, and/or where the distances between engagement member 32b and anchor 54a, and between engagement member 32b and anchor 54b are unequal, the corresponding groove(s) may be configured to allow the corresponding shape memory member(s) 12, 14 to slide therein as its length changes and the load 20 undergoes oscillating movement. In embodiments where such distances are substantially equal, the corresponding shape memory member 12, 14 may be fixed to the corresponding engagement member 32a, 32b (e.g., at a mid-point along the corresponding length thereof).

As illustrated in FIG. 3A, first shape memory member 12 may operatively interconnect via engagement member 32a to shaft member 30 at a location offset from pivot axis AA so as to define a first moment arm I₁. Similarly, second shape memory member 14 may operatively interconnect via engagement member 32b to shaft member 30 at a location offset from pivot access AA so as to define a second moment arm I₂. In the illustrated arrangement, moment arms I₁ and I₂ are substantially equal. Arrangements may be implemented in which moment arms I₁ and I₂ are not equal.

In FIGS. 2A and 3A, the first shape memory member 12 has been actuated, e.g., heated, so as to cause the first shape memory member 12 to shrink in length and thereby rotate shaft member 30 in a first direction (e.g., clockwise) by y₁ degrees. As noted, first shape memory member 12 may be actuated during a first time period that is at least partially non-overlapping with a second time period during which second shape memory member 14 is actuated. In this regard, actuation of first shape memory member 12 may function to apply a tensile force to second shape memory member 14 so as to facilitate a return of shape memory member 14 to an extended state (e.g., in conjunction with its austenitic-to-martensitic phase transformation after actuation).

In FIG. 3B, the second shape memory member 14 has been actuated (e.g., heated) so as to cause the second shape memory member 14 to shrink in length and thereby rotate shaft member 30 in a second direction (e.g., counterclockwise) by y₂ degrees. In arrangements in which second shape memory member 14 is actuated in at least partially offset timed relation to actuation of the first memory shape member 12, the actuation of the second shape memory member 14 may function to apply a tensile force to the first shape memory member 12 so as to facilitate a return of first shape memory member 12 to an extended state (e.g., in conjunction with its austenitic-to-martensitic phase transformation after actuation).

Referring again to FIGS. 1 and 2A, portions of first shape memory member 12 extend away from engagement member 32a and load 20 to define an included angle of x₁ degrees therebetween. Similarly, portions of second shape memory member 14 extend away from engagement member 32b and load 20 to define an included angle of x₂ degrees therebetween. As may be appreciated, included angle x₁ increases and included angle x₂ decreases during actuation of first shape memory member 12, and included angle x₂ increases and included angle x₁ decreases during actuation of second shape memory member 14. The angular configurations of first shape memory member 12 and second shape memory member 14 illustrated in FIG. 1 facilitate pivoting movement of load 20 across a relatively large angular range of y₁+y₂ degrees (see FIGS. 3A and 3B). In this regard, where the shape memory members 12, 14 are varied in length of about 1% to 5% (e.g., 4%) and where the angles x₁ and x₂, in a neutral or “home” position (e.g., with the load 20 in a horizontal position), are about 100 to 170 degrees, the total angular range of y₁+y₂ degrees may be on the order of about 50-60 degrees. The same total angular range may be achieved in another embodiment by, for example, making the angles x₁ and x₂ in the home position larger and correspondingly decreasing the variation in length of the shape memory members 12, 14. Such a variation may result in higher stress on the shape memory members 12, 14. In another variation, making the angles x₁ and x₂ in the home position smaller and correspondingly increasing the variation in length of the shape memory members 12, 14, may increase the linearity between the change in length of the shape memory members 12, 14 and the change in angle of the load 20. The location of the fixed ends of the shape memory members 12, 14 on the first and second end pieces 42a, 42b relative to the where the shape memory members 12, 14 interface with the engagement members 32a, 32b may be adjusted to, for example, provide a maximum force imparted on the engagement member 32a, 32b by the shape memory members 12, 14 at a selected point in the motion cycle of the load 20. The location of the fixed ends of the shape memory members 12, 14 may also be selected such that a particular overall volume of space taken up by the actuator 10 may be achieved. Thus, for a particular application, the actuator 10 may be configured to achieve a certain size, while in another configuration, the actuator may be configured to achieve a certain linearity, while in another configuration, a particular angular range of y₁+y₂ degrees may be achieved. In one example, the actuator may be configured such that it occupies a volume of space defined by an imaginary cylinder created by rotating the load 20 through 360 degrees about the pivot axis AA. In such an example, the overall diameter of the actuator 10 may be determined by the load 20 size as opposed to the size of the mechanisms used to drive the load 20. In this regard, load 20 size (e.g., length, width, thickness) may be a factor in the configuration of the shape memory members 12, 14.

Returning to the embodiment of FIGS. 1, 2A, 3A and 3B, actuation of the first shape memory member 12 may be realized via the provision of energy signals to anchors 52a and 52b, which may be electrically interconnected to shape memory member 12. In this regard, anchors 52a and 52b may serve as connector blocks facilitating electrical interconnection to shape memory member 12. Similarly, actuation of the second shape memory member 14 may be realized via the provision of energy signals to anchors 54a and 54b, which may be electrically interconnected to shape memory member 14. For example, anchors 52a, 52b, and 54a, 54b may be interconnected via electrical signal lines to an electrical energy source comprising logic to provide electrical signals to anchors 52a, 52b and 54a, 54b (and therefore to shape memory members 12, 14) in offset, timed-relation, wherein such electrical signals may vary in magnitude according to a predetermined algorithm. Such predetermined algorithm may be established to realize a relatively constant angular velocity of load 20 as it pivots, or rotates, about pivot axis AA in a oscillating manner. Alternatively, a predetermined algorithm may be established to realize other desired motion profiles for the load 20. Indeed, by altering the algorithms used to drive shape memory members, the motion profile of any of the embodiments discussed herein may be adjusted as desired.

Magnets may be used under various circumstances to control the motion of the load 20. For example, as shown in FIG. 3C, a magnet 62 may be positioned at or near the end of travel of the engagement member 32a. In such a configuration, the engagement members 32a, 32b may be made from a magnetizable (e.g., ferrous) material. Alternatively, the engagement members 32a, 32b may be made from a non-magnetizable material and one or more magnetizable members may be fixedly interconnected to the engagement members 32a, 32b to enable the magnet 62 and a second magnet 60 to impart a magnetic force on the engagement members 32a, 32b. The magnet 62 may impart an attractive force on the engagement member 32a, thus reducing the elastic tension necessary in the first shape memory member 12 to achieve the end of travel position shown in FIG. 3C. Such an arrangement may also reduce the level of heating of the shape memory member 12 necessary to achieve the end of travel position. The second magnet 60 may be correspondingly positioned to have a similar effect on the load 20 at the other end of travel position. In a variation of the embodiment illustrated in FIG. 3C, the magnet 62 may be positioned such that it comes in direct contact with the engagement member 32a at the end of travel position. Such a configuration may serve to positively determine the position of the load 20 (i.e., by driving the engagement member 32a into contact with the magnet 62, the position of the load 20 will be known). Moreover, such a configuration may be used to provide a force capable of holding or assisting in holding the position of the load 20 at the end of travel for a predetermined length of time. In another variation, a non-ferrous spacer (not shown) may be fitted to the magnet 62 (or alternatively to the engagement member 32a) such that the spacer serves as a hard stop to the motion of the engagement member 32a (thus providing a positive determination of the position of the load 20), but does not allow magnet 62 to come into direct contact with the engagement member 32a.

In another example of magnetic assist shown in FIG. 3D, a pair of like-pole magnets 66, 70 may be positioned such that they impart a repulsive force on each other as the load 20 approaches the end of travel position shown in FIG. 3D. Such a configuration may assist in decelerating the load 20 and may be particularly applicable to relatively high speed and/or high load mass applications that may benefit from assisted deceleration. A similarly configured pair of like-pole magnets 64, 68 positioned to have a similar effect on the load 20 at the other end of travel position may be used.

The above-described magnets may be permanent magnets and/or electromagnets. Where the magnets are electromagnets, they may be actively controlled to assist in providing a desired motion profile. Any other embodiment described herein may use magnets as described above to assist in the control of the motion of the loads. In embodiments utilizing magnets, the various parts that interface with the magnets may be shaped to provide particular performance characteristics. For example, the engagement members 32a, 32b of FIG. 3C may have a square cross section (as opposed to the circular cross section shown in FIG. 1) such that a flat surface is presented to the magnets 60, 62.

In an alternative arrangement of the components of the embodiment of FIG. 1, the ends of the shape memory members 12, 14 may be fixedly interconnected to the load 20 in a manner similar to how the ends of the shape memory members 12, 14 are fixedly attached to the first and second end pieces 42a, 42b in FIG. 1. In such an embodiment, the engagement members or equivalent structure may be fixedly (relative to the outer shell 42c) disposed below (i.e., below when in the orientation shown in FIG. 1) the load 20 such that the shape memory members 12, 14 may each have a first end fixedly interconnected to the load 20 at one end of the load 20, a second end fixedly interconnected to the load 20 at the other end of the load 20, and a central portion positioned partially about the fixedly disposed engagement members or equivalent structure.

In an additional alternative arrangement of the components of the embodiment of FIG. 1, the actuator 10 may include additional shape memory members to provide redundancy in the case of a failure of one or both of the shape memory members 12, 14. For example, an additional shape memory member, similarly configured to shape memory member 12, may be disposed such that it is operable to produce the same motion of the load 20 as shape memory member 12. In this regard, the additional shape memory member may be disposed generally parallel to shape memory member 12. In one embodiment, the additional shape memory member may be actuated in tandem with the shape memory member 12. Another shape memory member may be disposed and/or actuated relative to shape memory member 14 in a similar manner. Consequently, in such an arrangement, if one or both of the shape memory members 12, 14 were to fail, the redundant shape memory members could be employed to produce the reciprocating motion of the load 20.

FIG. 2B illustrates the shaft member 30 and engagement members 32a, 32b in the same orientation as FIG. 2A. In the embodiment of FIG. 2B, the shape memory members 12, 14 and corresponding elastically deformable members 53a, 53b, 55a, 55b and anchors 52a, 52b, 54a, 54b of FIG. 2A have been replaced with helically wound shape memory members 16, 18 and anchor members 22, 24. The helically wound shape memory members 16, 18 may be operable to achieve a higher percentage of reduction in length (e.g., along a longitudinal axis of helically wound coils) as compared to the non-helically wound shape memory members 12, 14. Thus, as illustrated in FIG. 2B, the helically wound shape memory members 16, 18 may be disposed generally perpendicular to the ends of the engagement members 32a, 32b to affect oscillating, pivotal movement of the shaft member 30 similar to that created by shape memory members 12, 14. Moreover, the helically wound shape memory members 16, 18 may be operable to produce such motion within a similar volume of space (e.g., within the enclosure 40 of FIG. 1). The anchor members 22, 24 may include elastically deformable members. Moreover, additional helically wound shape memory members may be used to provide redundancy similar to as described above with reference to additional shape memory members 12, 14.

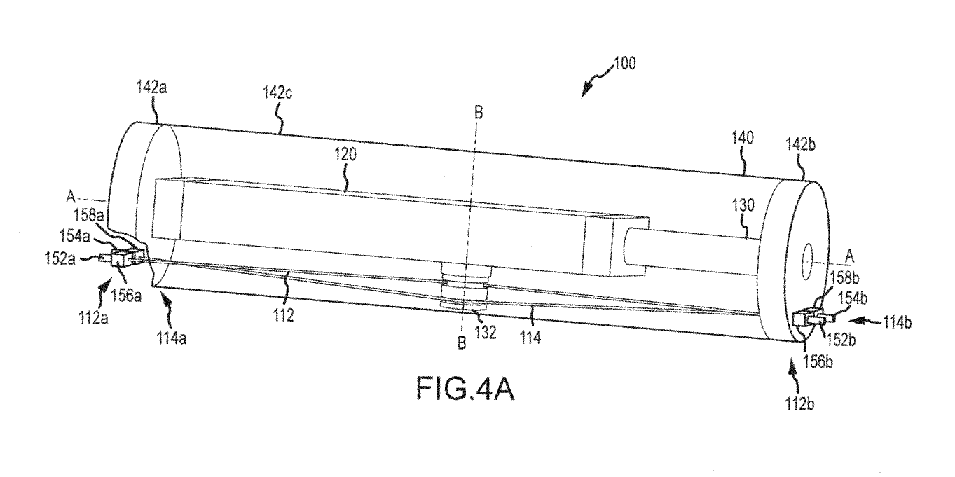

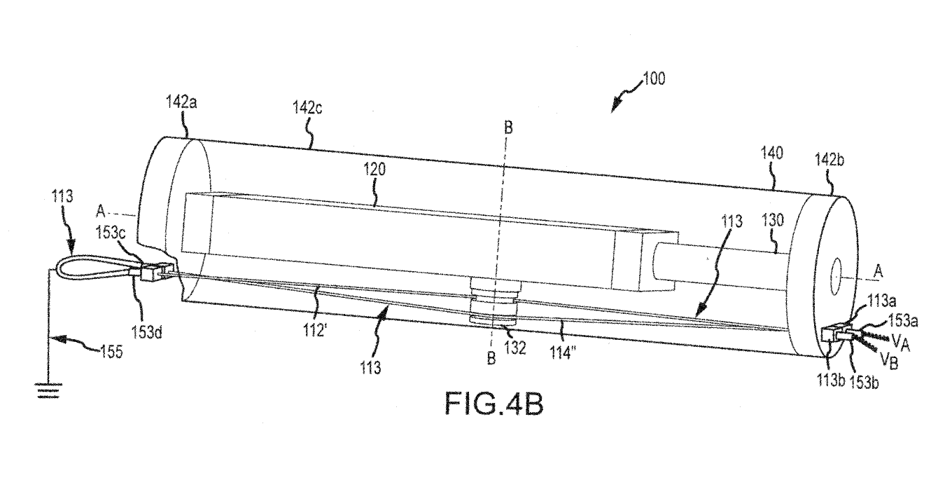

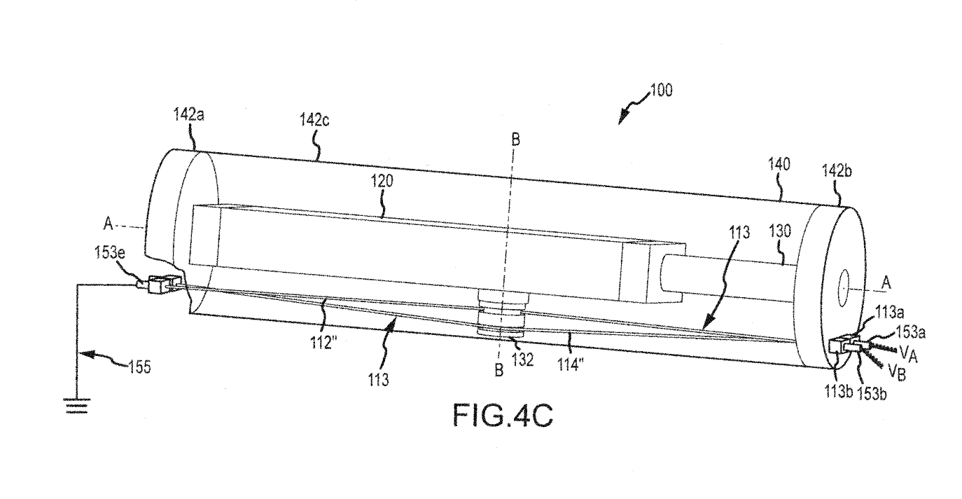

FIG. 4A illustrates another embodiment of an actuator 100 comprising a first shape memory member 112 and a second shape memory member 114 that are actuatable to affect oscillating, pivotal movement of a load 120 about a pivot axis AA. Pivot axis AA may be defined by a shaft member 130 that is journaled at each end and rotatable relative to an enclosure 140. The enclosure 140 includes a first end piece 142a, a second end piece 142b, and an outer shell 142c (shown as transparent in FIG. 4A). As illustrated, load 120 may be supportably mounted to the shaft member 130 for pivoting movement therewith.

The first and second shape memory members 112, 114 may each comprise a length of shape memory wire or any other appropriate shape memory form (e.g., a shape memory ribbon, a multiple element member such as a multiple filament wire, a coil, a helically wound strand) and may be heated in at least partially offset, timed-relation to yield corresponding martensitic-to-austenitic phase transformations and corresponding reductions (e.g., shrinkage) in the length of each wire. In turn, such alternating length reductions causes shaft member 130 to pivot, or rotate back and forth, thereby causing load 120 to pivot back and forth about pivot axis AA in an oscillating manner.

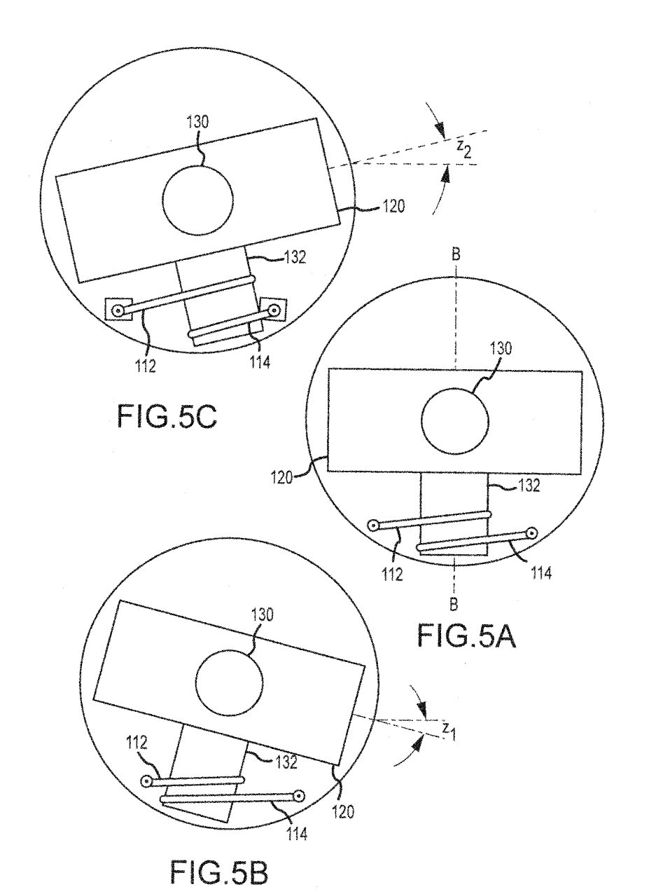

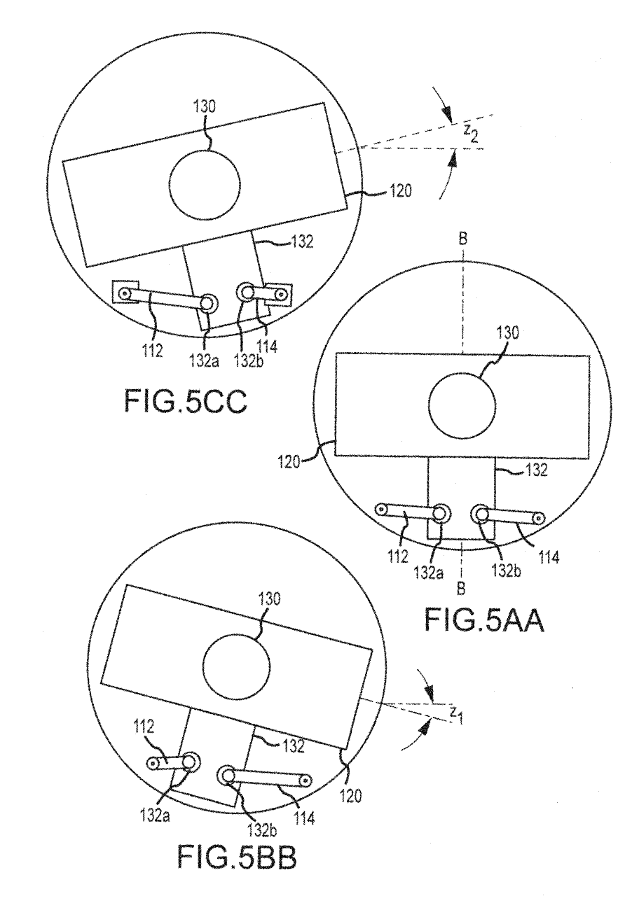

As shown in FIG. 4A, first shape memory member 112 may be fixedly interconnected at a first end 112a to an anchor 152a interconnected to enclosure 140 via an elastically deformable member 156a, and first shape memory member 112 may be fixedly interconnected at a second end 112b to an anchor 152b interconnected to enclosure 140 via an elastically deformable member 156b. Each of anchors 152a and 152b may be disposed on a common side of a vertical plane that contains both pivot axis AA and an axis BB, which, when the load 120 is in a “home” position (as shown in FIG. 4A), lies along a engagement member 132 that extends downwardly away from shaft member 130 in fixed relation thereto (see FIG. 5A). The second shape memory member 114 may be interconnected at a first end 114a to an anchor 154a interconnected to the enclosure 140 via an elastically deformable member 158a, and second shape memory member 114 may be fixedly interconnected at a second end 114b to an anchor 154b interconnected to the enclosure 140 via an elastically deformable member 158b. Each of the anchors 154a and 154b may be disposed on a common side of the vertical plane, defined by axes A-A and B-B, opposite to the side on which anchors 152a, 152b are disposed. Alternatively, only a single elastically deformable member (e.g., elastically deformable members 156a, 158a) may be interconnected to each shape memory member 112, 114, or no elastically deformable member may be employed.

As further illustrated in FIG. 4A, first shape memory member 112 and second shape memory member 114 are disposed to operatively interconnect with shaft member 130 via engagement with opposing sides of the engagement member 132. More particularly, first shape memory member 112 engages a side of engagement member 132 that faces away from the side of the engagement member 132 on which anchors 152a, 152b are disposed. Conversely, second shape memory member 114 engages a side of engagement member 132 that opposes the side of the engagement member 132 engaged by first shape memory member 112 and that faces away from the side of the engagement member 132 on which anchors 154a, 154b are disposed.

It will be appreciated that, as shown in FIG. 4A, the first and second shape memory members 112, 114 are not configured such that they interface with the engagement member 132 at the same distance away from the load 120. Thus, the first and second shape memory members 112, 114 may not symmetrically act upon the engagement member 132. In a variation of the actuator 100 of FIG. 4, the first and second shape memory members 112, 114 may be configured such that they each interface with the engagement member 132 at a common distance from the load 120. In such a configuration, symmetry may, for example, be achieved by symmetrically adjusting the positions of the anchors 152a, 152b, 154a, 154b such that the first and second shape memory members 112, 114 do not interfere with each other during pivoting of the load 120.

FIG. 4B illustrates a modified embodiment of the actuator 100 shown in the FIG. 4A embodiment. In relation to the FIG. 4A embodiment it was noted that the first and second shape memory members 112, 114 may comprise lengths of shape memory wire. FIG. 4A illustrates physically-separate first and second shape memory members 112, 114. In the FIG. 4B embodiment, the first and second shape memory members 112′, 114′ may be defined by separate segments, or lengths, of a continuous shape memory wire 113. By way of example, the shape memory alloy wire 113 may be crimped at a first end 113a to a crimp anchor 153a and crimped at a second end 113b to a crimp anchor 153b. Further, the shape memory alloy wire 113 may be crimped at crimp anchor 153c to define a wire segment corresponding with first shape memory member 112′ (i.e., between crimp anchor 153a and 153c), and crimped at crimp anchor 153d to define the second shape memory member 114′ (i.e., between crimp anchor 153b and 153d). In this arrangement, the shape memory alloy wire 113 may be electrically interconnected to a common electrical ground 155 (e.g., between crimp anchors 153c and 153d). As illustrated, the first end 113a of the shape memory alloy wire 113 may be electrically interconnected to a first electrical drive signal source V_{A}, and the second end 113b may be electrically interconnected to a second electrical drive signal source V_{B}. The first and second electrical drive signal sources V_{A}, V_{B} may be alternately operated for actuation of first and second shape memory members 112′, 114′, respectively.

FIG. 4C illustrates a modified version of the embodiment of FIG. 4B. As illustrated, a shape memory alloy wire 113 may be crimped at a single crimp anchor 153c. In such arrangement, a first shape memory member 112″ and second shape memory member 114″ may define a V-shaped configuration between the first end piece 142a and engagement member 132. The crimp anchor 153c may electrically interconnect to the common electrical ground 155.

The first and second shape memory members 112, 114 of FIG. 4A, the first and second shape memory members 112′, 114′ of FIG. 4B, and the first and second shape memory members 112″, 114″ of FIG. 4C, may each be in the form of shape memory wire lengths. In one approach, such shape memory wire lengths may comprise physically-separate first and second wires (e.g., first and second shape memory members 112, 114). In another approach, such shape memory wire lengths may be defined by different segments of a continuous shape memory wire (e.g., first and second shape memory members 112′, 114′ and first and second shape memory members 112″, 114″).

Reference is now made to FIGS. 5A, 5B and 5C which illustrate the operative interface between the first shape memory member 112 and shaft member 130 via engagement member 132, and between the second shape memory member 114 and shaft member 130 via engagement member 132. In FIG. 5A, actuator 100 is shown in a “home” position, e.g., prior to actuation with shape memory members 112, 114 each in a martensitic state and with the load 120 disposed in a position that is substantially centered between the two extremes of the load's 120 range of oscillating movement. In FIG. 5B, the first shape memory member 112 has been actuated, e.g., heated, so as to cause the first shape memory member 112 to shrink in length and thereby rotate engagement member 132, shaft member 130 and load 120 in a first direction (e.g., clockwise) by z₁ degrees. As noted, first shape memory member 112 may be actuated during a first time period that is at least partially non-overlapping with a second time period during which second shape memory member 114 is actuated. In this regard, actuation of first shape memory member 112 may function to apply a tensile force to second shape memory member 114 so as to lengthen second shape memory member 114 (e.g., in conjunction with an austenitic-to-martensitic phase transformation after actuation).

In FIG. 5C, the second shape memory member 114 has been actuated (e.g., heated) so as to cause the second shape memory member 114 to shrink in length and thereby rotate engagement member 132, shaft member 130 and load 120 in a second direction (e.g., counterclockwise) by z₂ degrees. In arrangements in which second shape memory member 114 is actuated in at least partially offset timed-relation to actuation of the first shape memory member 112, the actuation of the second shape memory member 114 may function to apply a tensile force to the first shape memory member 112 so as to lengthen first shape memory member 112 (e.g., in conjunction with an austenitic-to-martensitic phase transformation after actuation).

FIGS. 5AA, 5BB and 5CC illustrate a modified arrangement of the embodiment shown in FIG. 4A, in corresponding relation to the views of FIGS. 5A, 5B and 5C. As illustrated, engagement member 132 is provided with apertures 132a, 132b for receiving first and second shape memory members 112, 114 therethrough, respectively.

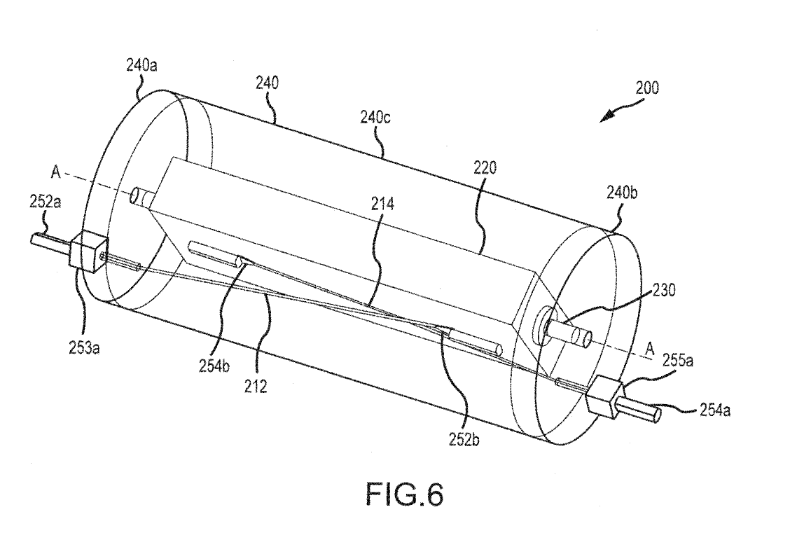

FIG. 6 illustrates another embodiment of an actuator 200 comprising a first shape memory member 212 and a second shape memory member 214 that are actuatable to affect oscillating, pivotal movement of a load 220 about a pivot axis AA. Pivot axis AA may be defined by a shaft member 230 that is journaled at each end and rotatable relative to an enclosure 240. The enclosure 240 includes a first end piece 240a, a second end piece 240b, and an outer shell 240c (all shown as transparent in FIG. 6).

As illustrated, load 220 may be supportably mounted to the shaft member 230 for pivoting movement therewith. The first and second shape memory members 212, 214 may each comprise a length of shape memory wire and may be heated in at least partially offset timed-relation to yield corresponding martensitic-to-austenitic phase transformations and corresponding reductions (e.g., shrinkage) in the length of each wire. In turn, such alternating length reductions cause shaft member 230 to rotate back and forth, thereby causing load 220 to pivot back and forth about pivot axis AA in an oscillating manner. As shown, first shape memory member 212 may be fixedly interconnected at a first end to an anchor 252a interconnected to enclosure 240 via an elastically deformable member 253a, and first shape memory member 212 may be fixedly interconnected at a second end to an anchor 252b fixedly interconnected to a bottom surface of load 220. Similarly, second shape memory member 214 may be fixedly interconnected at a first end to an anchor 254a interconnected to the enclosure 240 via an elastically deformable member 255a and second shape memory member 214 may be fixedly interconnected at a second end to an anchor 254b fixedly interconnected to the bottom surface of load 240. Alternatively, anchor 252b may be fixedly interconnected to an elastically deformable member (not shown) that in turn is interconnected to the load 220, and anchor 254b may be fixedly interconnected to another elastically deformable member (not shown) that in turn is interconnected to the load 220. In such an alternate embodiment, the elastically deformable members 253a, 253b are optional.

Anchors 252a and 254a may be located at opposing ends of the enclosure 240 and on opposite sides of a plane that includes the pivot axis AA and is perpendicular to the plane of the load 220 when the load is in a “home” position, e.g., prior to actuation with shape memory members 212, 214. Further, anchors 252b and 254b may be disposed at offset locations relative to the plane when the load is in a “home” position. In an embodiment, anchor 252a and anchor 252b may be disposed on opposite side of the plane when the load is in a “home” position, and anchor 254a and anchor 254b may be disposed on opposite sides of the plane when the load is in a “home” position. In this regard, when the load is in the “home” position each of the shape memory members 212, 214 may cross the plane as they extend from their respective anchors 252a, 254a on the enclosure 240 to their respective anchors 252b, 254b on the load 220.

In FIG. 6, first shape memory member 212 has been actuated so as to cause shaft member 230 to rotate and load 220 to pivot in a clockwise direction (as viewed from the right side of the actuator 200 as shown in FIG. 6). As may be appreciated, upon actuation of the second shape memory member 214 and deactuation of first shape memory member 212 the shaft member 230 may be rotated and load 220 may be pivoted by the second shape memory member 214 in a counterclockwise direction.

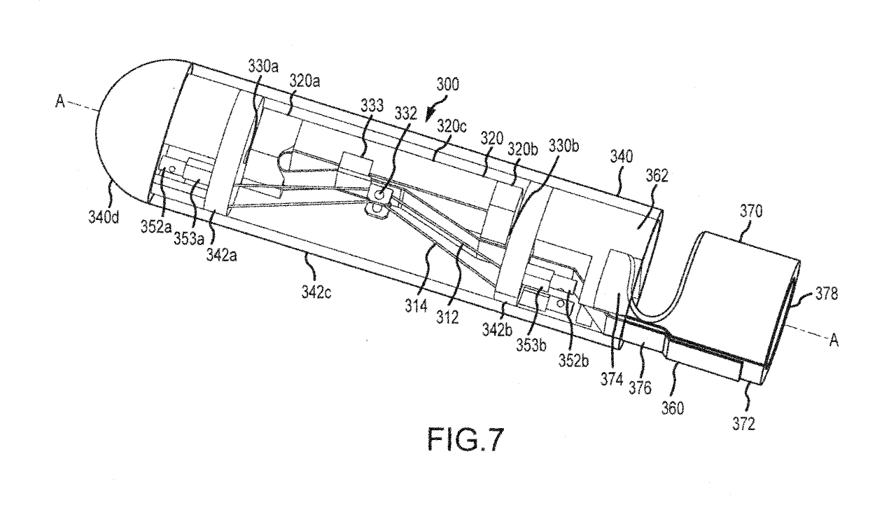

FIG. 7 illustrates an actuator 300, similar to that shown in the embodiment of FIG. 1, configured for use in an imaging catheter application. More particularly, FIG. 7 illustrates actuator 300 comprising a first shape memory member 312 and a second shape memory member 314 that are actuatable to effect oscillating, pivotal movement of a load 320 about a pivot axis AA. The pivot axis AA is shown in FIG. 7 to coincide with a central longitudinal axis of the actuator 300. Alternatively, in an embodiment, the pivot axis AA may be offset from the central longitudinal axis of the actuator 300. The load 320 comprises three portions, a first end block 320a, a second end block 320b, and an active block 320c fixedly interconnected to and disposed between the end blocks 320a, 320b. The active block 320c may be in the form of an ultrasound transducer array. Pivot axis AA may be defined by collinear shaft members 330a, 330b which are journaled and rotatable relative to an enclosure 340. In turn, load 320 may be supportably mounted to the shaft members 330a, 330b for pivoting movement therewith. The enclosure 340 includes a first end piece 342a, a second end piece 342b, and an outer shell 342c (shown as transparent in FIG. 7). The enclosure 340 further includes an end cap 340d, which may be rounded to facilitate movement through a body. The first end piece 342a and the second end piece 342b, and therefore the pivot axis AA may be fixed relative to the enclosure 340.

Where the active block 320c is an ultrasound transducer array, the ultrasound transducer array may be operable to transmit acoustic signals that may be used to generate an image of a two-dimensional plane extending from a length dimension of the ultrasound transducer array. By affecting oscillating motion of the ultrasound transducer array using the shape memory members 312, 314, the two-dimensional imaging plane of the ultrasound transducer array may be swept through a three-dimensional volume thus enabling creation of three dimensional images. Such three dimensional images may be real-time (4D).

The first and second shape memory members 312, 314 may be configured similarly to the first and second shape memory members 12, 14 of FIG. 1. As will be appreciated, alternating length reductions of the first and second shape memory members 312, 314 causes the load 320 to pivot back and forth about pivot axis AA in an oscillating manner.

The first shape memory member 312 may be fixedly interconnected at a first end to an anchor 352a. The anchor 352a may be interconnected to an elastically deformable member 353a, which in turn is interconnected to first end piece 342a. First shape memory member 312 may be fixedly interconnected at a second end to an anchor 352b. Likewise, the anchor 352b may be interconnected to an elastically deformable member 353b, which in turn is interconnected to second end piece 342b. Thus, first shape memory member 312 may be configured similarly to first shape memory member 12 of FIG. 1. In a similar fashion, second shape memory member 314 may be configured similarly to second shape memory member 14 of FIG. 1.

The first shape memory member 312 may be operatively interconnected to load 320 via a cross shaft 332. The cross shaft 332 may in turn be fixedly interconnected to a cross shaft bracket 333 that may be fixedly interconnected to the load 320. The cross shaft 332 may be disposed in an orientation and position similar to that of the engagement members 32a, 32b of FIG. 1.

The first and second shape memory members 312, 314 may be disposed along the cross shaft 330 in a manner similar to how first and second shape memory members 12, 14 of FIG. 1 interface with engagement members 32a, 32b. In this regard, oscillating movement of load 320 via actuation of the first and second shape memory members 312, 314 may be achieved in a manner similar to that as described with respect to FIG. 1.

An electrical interconnection member 360 may be electrically interconnected to the active block 320c. For example, the electrical interconnection member 360 may be a multiple conductor member that provides electrical interconnections to the active block 320c. The electrical interconnection member 360 may be routed through second end piece 342b, between the cross shaft 332 and the active block 320c, to the end of the active block 320c proximate to the first end piece 342a. In this regard, the portion of the electrical interconnection member 360 disposed between the second end piece 342b and the cross shaft 332 may be operable to flex while maintaining an electrical connection to the active block 320c. By way of example, the electrical interconnection member 360 may comprise flexboard (a flexible/bendable electrical member or plurality of members). In an embodiment, the flexboard may be disposed in a service loop or clockspring arrangement. Such a clockspring arrangement may be disposed within the actuator 300. For example, the end member 362 may house the clockspring arrangement.

An end member 362 may be interconnected to the actuator 300 at an end opposite from the end cap 340d. The end member 362 may provide a structure that is capable of interfacing with external components, such as components of a catheter body, to enable the actuator 300 to be interconnected to other structures, such as a catheter body. The end member 362 may also serve to seal the actuator 300 such that an enclosed volume is defined by the end member 362, the end cap 340d and the outer shell 342c.

The actuator 300 may be interconnected to a distal end of a catheter body such that the actuator 300 is fixed relative to the distal end of the catheter body. In another arrangement, actuator 300 may be interconnected to a distal end of a catheter body such that the actuator is rotatably positionable relative to the distal end of the catheter body. For example, the actuator 300 may be interconnected to a drive member that extends along the length of the catheter body from a distal end to a proximal end thereof, wherein rotation of a proximal end of the drive member causes actuator 300 to rotate (e.g., rotate about an axis corresponding with a longitudinal or central axis of the catheter body at the distal end thereof).

Alternatively, and as illustrated in FIG. 7, the actuator 300 may be interconnected to a hinge 370. The hinge 370, in turn, may be interconnected to a distal end of a catheter body such that a portion of the hinge 370 is fixed relative to the distal end of the catheter body. The hinge 370 may include a catheter interface portion 372 operable to interconnect to a catheter body, an actuator interface portion operable to interconnect to the actuator 300, and a bendable portion 376 operable to allow relative angular movement between the actuator interface portion 374 and the bendable portion 376, thus allowing relative angular movement between the actuator 300 and a distal end of a catheter body. In this regard, the actuator 300 may be selectively positionable across a range of angles relative to a catheter body (e.g., relative to a longitudinal or central axis of a catheter body at a distal end thereof). As noted, the end member 362 may also serve to seal the actuator 300 or alternatively and as shown in FIG. 7, the end member 362 and the actuator interface portion may serve together to seal the actuator 300. The catheter interface portion 372 may include a central lumen 378 that may align with a lumen in a catheter.

Where the active block 320c is in the form of an ultrasonic transducer array, the ultrasonic transducer array may include an acoustic coupling medium attached to an active face of the ultrasonic transducer array. The acoustic coupling medium may comprise a hydrogel capable of absorbing liquid. By way of example, such acoustic coupling medium may be provided for acoustic coupling to the active face of the ultrasonic transducer array.

The enclosures 40 (FIG. 1), 140 (FIG. 4), 240 (FIG. 6) and 340 (FIG. 7) may define enclosed volumes. The enclosed volumes may contain a fluid therein. The fluid may be a liquid. In this regard, the loads and the first and second shape memory members may be immersed within the fluid within the enclosed volume. With respect to actuator 300 of FIG. 7, where the active block 320c is in the form of an ultrasonic transducer array, the fluid may serve to acoustically couple the ultrasound transducer array to the outer shell 342c. In this regard, the material of the outer shell 342c may be selected to correspond to (e.g., closely match) the acoustic impedance and/or the acoustic velocity of the fluid of the body of the patient in the region where the actuator 300 is to be disposed during imaging. One or more ports and/or valves may be provided to facilitate the placement of fluid within the actuators. Where the fluid is a liquid, multiple ports and or valves may be used to further facilitate the removal of bubbles from the enclosed volumes.

Alternatively, the actuators may not include an enclosed volume as described above, and the interior of the actuators may be open to the surrounding environment. For example, the enclosure 340 of the actuator 300 may include holes or open portions (not shown) that would allow fluid to pass between the interior of the actuator 300 and the surrounding environment. In this regard, fluid from the body of the patient in the region where the actuator 300 is to be disposed during imaging (e.g., blood where imaging the heart) may be allowed to flow into the interior of the actuator 300.

In another alternative, a portion of the actuators may be disposed within an enclosed volume, while at least portion of the load is open to the surrounding environment. For example, the load 320 of the actuator 300 may be sealably interconnected about a periphery of the load 320 to the enclosure 340 (e.g., by a flexible bellows), wherein a sealed lower portion and an upper portion may be defined. The lower portion may include a fluid and shape memory members 212, 214. The upper portion of the enclosure 340 may include holes, wherein a face of the active block 320c (e.g., an ultrasound transducer array) may be exposed to the surrounding environment (e.g., blood in heart imaging applications).

The shape memory members described herein may include one or more layers of material wrapped about a core that includes a shape memory wire. Such layers may act as thermal insulation layers, electrical insulation layers, or a combination of thermal and electrical insulation layers. For example, shape memory members 312, 314 may include an inner core comprising a shape memory wire and thermal insulation layer of PTFE. Other exemplary materials that may be used to insulate include ePTFE, and high strength toughened fluoropolymer (HSTF). Some thermal insulation layers may be microporous. Microporous thermal insulation layers entrap air that desirably contributes to an increase in thermal resistance. However, some microporous thermal insulation materials may wet out with blood and other body fluids, which may generally reduce their thermal resistance. Hydrophobic materials may be used in the microporous thermal insulation layers to reduce and/or prevent such wetting. Hydrophobic materials such as fluoropolymers may serve this purpose. Alternatively, non-hydrophobic materials may be treated with a hydrophobic and/or oleophobic treatment to render them suitable for this purpose. Preferred thermal insulation materials may have a surface energy less than 50 dyn/cm². Others may have a surface energy less than 40 dyn/cm². Still others may have a surface energy less than about 30 dyn/cm².

The thermal insulation layer may serve to insulate the shape memory wire such that the rate of dissipation of heat from the shape memory wire may be advantageously selected. For example, by selecting a predetermined thickness of thermal insulation layer to achieve a predetermined level of insulation, the heat flow from the shape memory wire to the surrounding environment (e.g., fluid) while the shape memory wire is being heated may be advantageously controlled to achieve a desired response time and/or level of heat transfer. That is, by adding insulation to the shape memory wire, the amount of heat lost to the surrounding environment during the heating of the shape memory wire may be reduced (relative to a configuration without insulation) thus reducing the time and/or power needed to heat the shape memory wire to produce a desired length change. Moreover, by reducing the power needed to produce the desired length change, the overall heat transfer to the surrounding environment may be reduced (again, relative to a configuration without insulation). In applications such as catheters, such reduction of power and associated reduction of heat transferred to the surrounding environment (e.g., the body of a patient) may enable the catheter to remain within an acceptable temperature range (e.g., below a certain regulated threshold that may be mandated by, for example, the U.S. Food and Drug Administration and/or International Electrotechnical Commission international standard IEC60601) during operation of the actuator 300. In an exemplary embodiment, the thermal insulation layer may have a thermal conductance of between about 0.03 W/mK and 0.20 W/mK when measured at about 25° C. In another exemplary embodiment, the thermal insulation layer may have a thermal conductance of between about 0.05 W/mK and 0.08 W/mK when measured at about 25° C.

The thermal and/or electrical insulation layers discussed above may provide acceptable withstand voltage and/or hydrophobicity, or the shape memory members described herein may include an additional layer of material disposed outside of the thermal insulation layer to provide the desired characteristics. The additional layer may, for example, add to the withstand voltage of the shape memory members such that they have an overall dielectric withstand voltage of at least about 500 kV/m. The additional layers may, for example, comprise a hydrophobic material. Such additional layers of hydrophobic material may have a surface energy of less than about 50 dyn/cm². Others may have a surface energy less than 40 dyn/cm². Still others may have a surface energy less than about 30 dyn/cm². The hydrophobic material may, for example, include ePTFE.

Hydrophobic materials may be beneficial as the additional layer in that they may act as a barrier layer to allow underlying layers to remain relatively free of liquid and thus maintain their insulative properties. Where the hydrophobic materials are used as the only layer, their use may be beneficial in that they do not absorb liquid to a degree that their thermal conductivity is significantly altered. Other materials that provide the same benefits (e.g., capable as acting as a barrier and/or capable of retaining insulative properties while immersed in liquid) as such hydrophobic materials may be utilized. The thermal and/or electrical insulation layers may also provide a lubricious and/or low friction interface to facilitate smooth motion over and/or around other components in the actuator during motion.

With respect to the above-described layers disposed about the shape memory members, a first step in determining the configuration of the layers may be to select a desired time constant for the system and then select the specific materials to achieve that time constant. For example, a time constant may be selected such that the cooling of the shape memory members is as slow as possible while still meeting desired load pivoting rates. Thus power dissipation could be minimized. Similarly, a particular power dissipation may be selected to allow for a particular application, then a corresponding time constant may be selected to provide for a maximum load pivoting rate for a particular application based on allowed power dissipation.

The use of shape memory members to produce oscillating motion of a load as illustrated in FIGS. 1 through 7 may be beneficial in that such systems may be relatively small. For example, the actuator 300 may include an ultrasound transducer array (e.g., active block 320c) that may be pivoted in an oscillating manner to generate real-time 3D images (4D images) while having an outer diameter of 12 Fr or less (e.g., 10 Fr). The shape memory wire used in the shape memory members may be about 1 mil in diameter. In the embodiment of FIG. 7, the moment arms I₁ and I₂ may be about 1.0 mm.

The actuators described herein may further include an encoder and/or position detector (e.g., to detect a load at an end of travel and/or at the “home” position) capable of providing feedback as to the position of the load being actuated. Such encoders and/or position detectors may allow servo control systems to control the position of the load being actuated.

The actuators described herein may be capable of producing oscillating movement of the loads up to and exceeding 50 Hz. For example, the actuators may be employed to produce oscillating movement of the loads in the 1-50 Hz or 8-30 Hz ranges. Such movement may be steady state to, for example, move the load, in the form of an ultrasound transducer, to facilitate 4D images. The actuators described herein may also be employed to move the loads relatively quickly (e.g., at the 50 Hz rate) to facilitate the capture of a 3D image during a single pivoting of the ultrasound transducer in a single direction. An image captured during such a single pivoting may provide a sharper “snapshot” of a volume of interest than would an image captured during relatively slower load movement. Such “snapshots” may be beneficial in imaging moving subjects, such as portions of a heart.

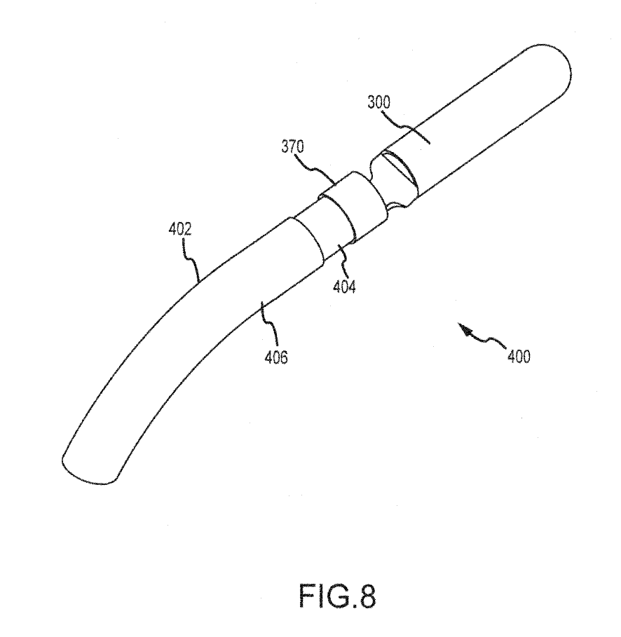

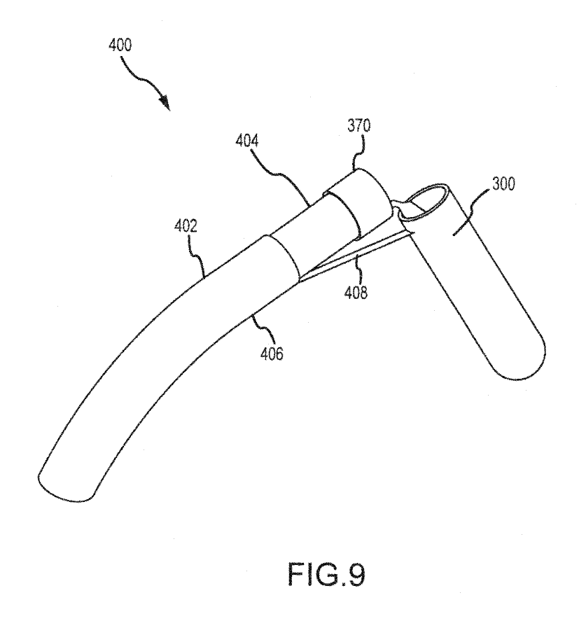

FIGS. 8 and 9 illustrate a distal end of a catheter assembly 400 that includes an elongate catheter body 402 that is connected by the hinge 370 to the actuator 300. FIG. 8 illustrates the actuator 300 that is a distal end portion of the catheter assembly 400 in a position where it is aligned with the distal end of the catheter body 402. FIG. 9 illustrates the actuator 300 in a position where it is deployed at about a +90 degree, forward-facing angle with respect to the end of the catheter body 402. For explanatory purposes only, an angular value (e.g., the +90 degree angle of displacement shown in FIG. 9) may be used herein to describe the amount of angulation of the actuator 300 with respect to a central axis of the catheter body 402 away from a position where the actuator 300 and catheter body 402 are aligned. A positive value will be used to describe an angulation where the actuator 300 is moved such that it is at least partially forward-facing (e.g., the active block 320c in the “home” position is facing forward), and a negative value will generally be used to describe an angulation where the actuator 300 is moved such that it is at least partially rearward-facing.

To reposition the actuator 300 from the position of FIG. 8 to the position of FIG. 9, an inner tube 404 of the catheter body 402 may be advanced relative to an outer tube 406 of the catheter body 402. By virtue of the actuator 300 being tethered to the outer tube 406 by a tether 408, the advancement may cause the actuator 300 to be angled in a positive direction. The tether 408 may be anchored to the actuator 300 on one end and to the outer tube 406 on the other end. The tether 408 may be operable to prevent the tether anchor points from moving a distance away from each other greater than the length of the tether 408. In this regard, through the tether 408, the actuator 300 may be restrainably interconnected to the outer tube 406. Similarly, where the tether 408 has adequate stiffness, retraction of the inner tube 404 relative to the outer tube 406 from the position shown in FIG. 8 may cause the actuator 300 to be angled in a negative direction. The inner tube 404 may include a lumen therethrough.

The tether 408 may be a discrete device whose primary function is to control the angular repositioning of the actuator 300. In another embodiment, the tether 408 may be a flexboard or other multiple conductor component that, in addition to providing the tethering function, electrically interconnects components within the actuator 300 with components within the catheter body 402 or elsewhere. In another embodiment, the tether 408 may be a wire or wires used to electrically interconnect one or more components (e.g., shape memory members 312, 314) within the actuator 300 to componentry external to the actuator 300.

FIGS. 8 and 9 illustrate a configuration where the hinge 370 is a living hinge. A live or living hinge is a compliant hinge (flexure bearing) made from a flexible or compliant material, such as polymer. Generally, a living hinge joins two parts together, allowing them to pivot relative to each other along a bend line of the hinge. Living hinges are typically manufactured by injection molding. Polyethylenes, polypropylenes, polyurethanes, or polyether block amides such as PEBAX® are possible polymers for living hinges, due to their fatigue resistance.

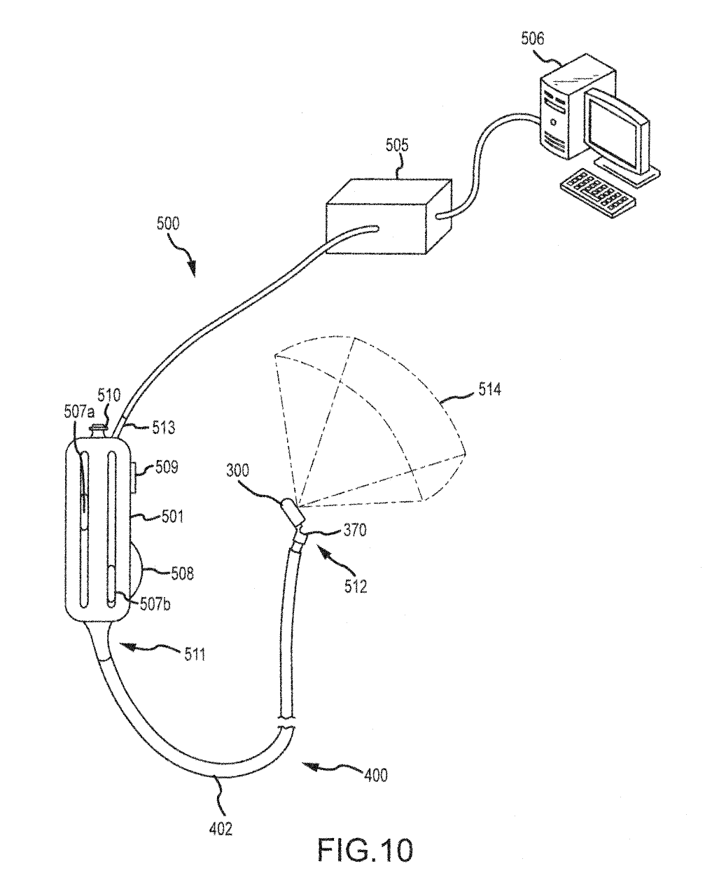

An application of the actuator 300 of FIGS. 7 through 9, where the active block 320c is in the form of an ultrasound transducer array, will now be described with reference to FIGS. 10 through 14.

FIG. 10 illustrates an ultrasound imaging system 500 suitable for real-time three dimensional (4D) imaging with a handle 501 and catheter 400. The catheter 400 includes the catheter body 402 interconnected to the actuator 300 via the hinge 370. The catheter body 402 may be flexible and capable of bending to follow the contours of a body vessel into which it is being inserted or track over a guidewire or through a sheath. The catheter body 402 may be steerable.

The ultrasound imaging system 500 may further include a controller 505 and an ultrasound console 506. The controller 505 may be operable to control the actuation of the shape memory members 312, 314 and thus the angular position of the ultrasound transducer array (i.e., active block 320c). The ultrasound console 506 may include an image processor, operable to process signals from the ultrasound transducer array, and a display device, such as a monitor. The various functions described with reference to the controller 505 and ultrasound console 506 may be performed by a single component or by any appropriate number of discrete components.

The handle 501 may be disposed at a proximal end 511 of the catheter 400. The user (e.g., clinician, technician, interventionalist) of the catheter 400 may control the steering of the catheter body 402, the angular repositioning of the actuator 300, and various other functions of the catheter 400. In this regard, the handle 501 includes two sliders 507a, 507b for steering the catheter body 402. These sliders 507a, 507b may be interconnected to control wires such that when the sliders 507a, 507b are moved relative to each other, a portion of the catheter body 402 may be curved in a controlled manner. Any other appropriate method of controlling control wires within the catheter body 402 may be utilized. For example, the sliders could be replaced with alternative means of control such as turnable knobs or buttons. Any appropriate number of control wires within the catheter body 402 may be utilized.

The handle 501 may further include an angular position controller 508. The angular position controller 508 may be used to control the angular position of the actuator 300 relative to a distal end 512 of the catheter body 402. The illustrated angular position controller 508 is in the form of a rotatable wheel, where a rotation of the angular position controller 508 will produce a corresponding angular position of the actuator 300. Other configurations of the angular position controller 508 are contemplated, including, for example, a slider similar to slider 507a.

The handle 501 may further include an actuator activation button 509. The actuator activation button 509 may be used to activate and/or deactivate the oscillating motion of the ultrasound transducer array within the actuator 300. The handle 501 may further include a port 510 in embodiments of the ultrasound imaging system 500 that include a lumen within the catheter body 402. The port 510 is in communication with the lumen such that the lumen may be used for conveyance of a device and/or material.

In use, the user may hold the handle 501 and manipulate one or both sliders 507a, 507b to steer the catheter body 402 as the catheter 400 is moved to a desired anatomical position. The handle 501 and sliders 507a, 507b may be configured such that the position of the sliders 507a, 507b relative to the handle 501 may be maintained, thereby maintaining or “locking” the selected position of the catheter body 402. The angular position controller 508 may then be used to angularly reposition the actuator 300 to a desired position. The handle 501 and angular position controller 508 may be configured such that the position of the angular position controller 508 relative to the handle 501 may be maintained, thereby maintaining or “locking” the selected angular position of the actuator 300. In this regard, the actuator 300 may be selectively angularly repositionable, and the catheter body 402 may be selectively steered, independently. Also, the angular position of the actuator 300 may be selectively locked, and the shape of the catheter body 402 may be selectively locked, independently. Such maintenance of position may at least partially be achieved by, for example, friction, detents, and/or any other appropriate means. The controls for the steering, angular repositioning, and motor may all be independently operated and controlled by the user.

The ultrasound imaging system 500 may be used to capture images of a three dimensional imaging volume 514 and/or capture 3D images in real-time (4D). The actuator 300 may be positioned by steering the catheter body 402, angularly repositioning the actuator 300, or by a combination of steering the catheter body 402 and angularly repositioning the actuator 300. Moreover, in embodiments with a lumen, the ultrasound imaging system 500 may further be used, for example, to deliver devices and/or materials to a selected region or selected regions within a patient.

The catheter body 402 may have at least one electrically conductive wire that exits the catheter proximal end 511 through a port or other opening in the catheter body 402 and is electrically connected to a transducer driver and image processor (e.g., within the ultrasound console 506).

Furthermore, in embodiments with a lumen, the user may insert an interventional device (e.g., a diagnostic device and/or therapeutic device) or material, or retrieve a device and/or material through the port 510. The user may then feed the interventional device through the catheter body 402 to move the interventional device to the distal end 512 of the catheter body 402. Electrical interconnections between the ultrasound console 506 and the actuator 300 may be routed through an electronics port 513 and through the catheter body 402.

One difficulty associated with the use of conventional ICE catheters is the need to steer the catheter to multiple points within the heart in order to capture the various imaging planes needed during the procedure. Catheter 400, incorporating the angularly repositionable actuator 300 with its oscillatingly pivotable ultrasound transducer array 320c therein, alleviates such difficulties associated with the use of conventional ICE catheters.

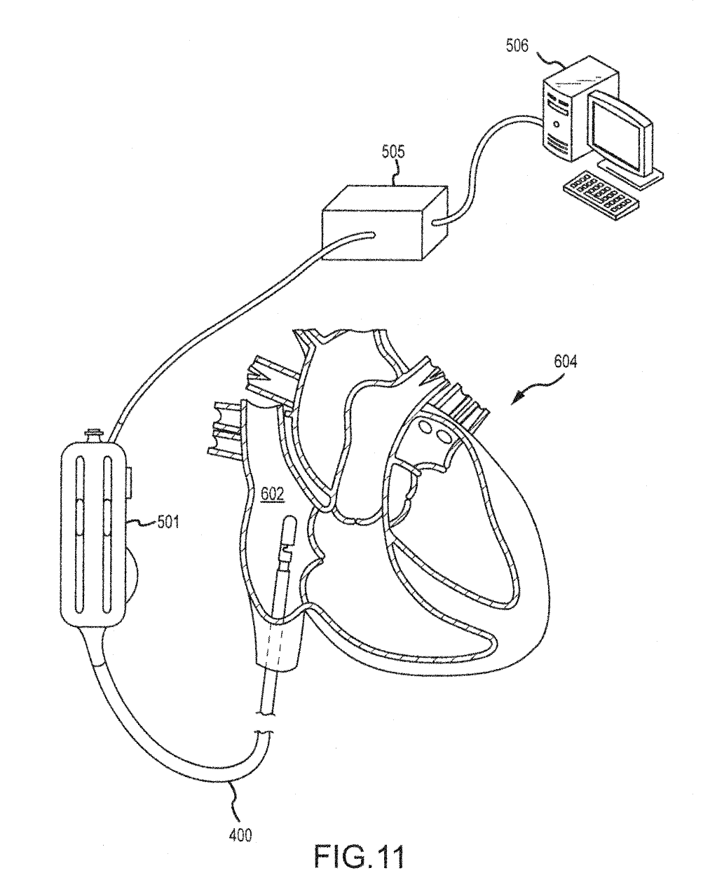

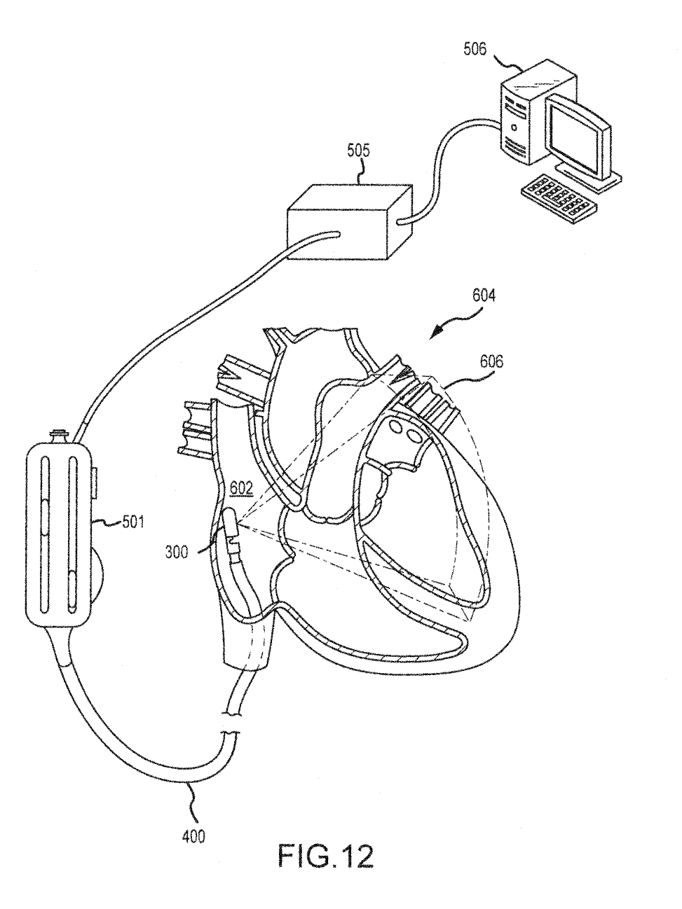

FIG. 11 shows placement of the catheter 400 for intracardiac echocardiography within the right atrium 602 of the heart 604. FIG. 12 shows placement of the catheter 400 within the right atrium 602 of the heart 604 after the catheter 400 has been repositioned (through steering of the catheter 400) to place the actuator 300 disposed at a distal end of the catheter 400 at a desired position. The clinician may establish and then set the catheter 400 position within the heart 604 by locking the catheter 400 position (locking mechanism on handle not shown). In this regard, once set, the catheter 400 position may remain substantially unchanged while the actuator 300 is angularly repositioned.

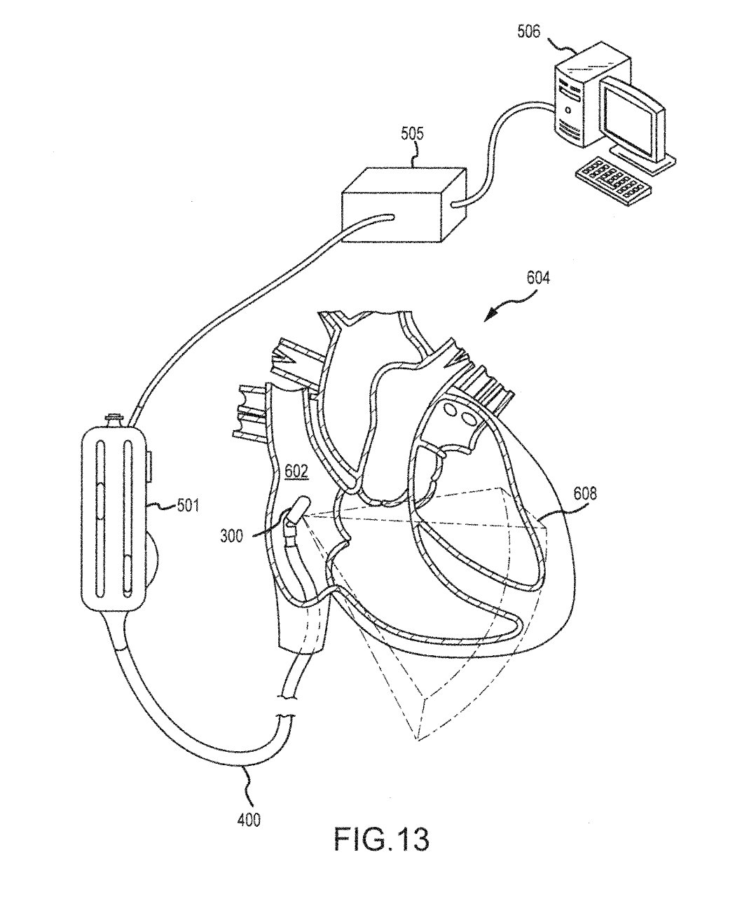

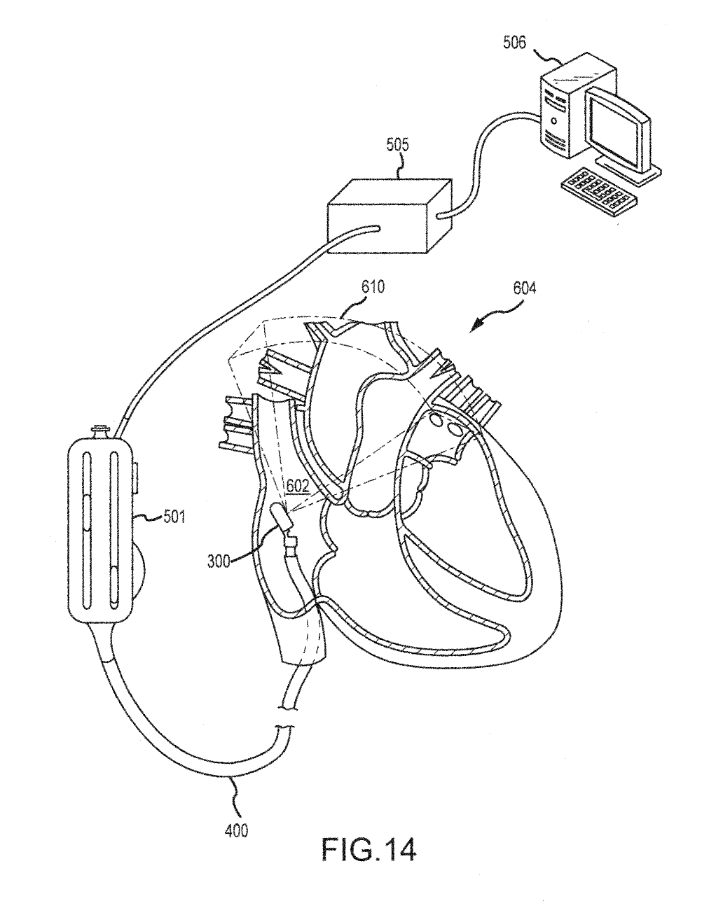

With the actuator 300 positioned as illustrated in FIG. 12, a volumetric image may be generated from a three dimensional volume 606 of a first portion of the heart 604. The clinician may then manipulate the actuator 300 orientation in order to capture the range of imaging volumes required. For example, FIG. 13 shows the actuator 300 angularly repositioned to a second position to capture a volumetric image of a three dimensional volume 608 of a second portion of the heart 604. FIG. 14 shows the actuator 300 angularly repositioned to a third position to capture a volumetric image of a three dimensional volume 610 of a third portion of the heart 604. Embodiments of actuator 300 described herein may be operable to achieve such positions and more within the right atrium 602 of the heart 604 that may have an intracardiac volume with cross dimension of about 3 cm. Volumetric images of such three dimensional volumes 606, 608, and 610 are obtainable by angularly repositioning the actuator 300 and operation of the actuator 300 to effectuate oscillating pivoting of the ultrasound transducer array while the distal end of the catheter 400 remains in the position as shown in FIG. 12.

Clinical procedures that may be performed with embodiments disclosed herein include without limitation septal puncture, septal occluder deployment, ablation, mitral valve intervention and left atrial appendage occlusion. A method for right atrial imaging utilizing embodiments may include advancing the catheter body 400 to the right atrium, steering the distal end 512 of the catheter body 400 to a desired position, operating the actuator 300 to effectuate movement of the ultrasound transducer array disposed therein, and while maintaining the fixed catheter body 400 position, angularly reposition the actuator 300 comprising the ultrasound transducer array about the hinge 370 to capture at least one image over at least one viewing plane.

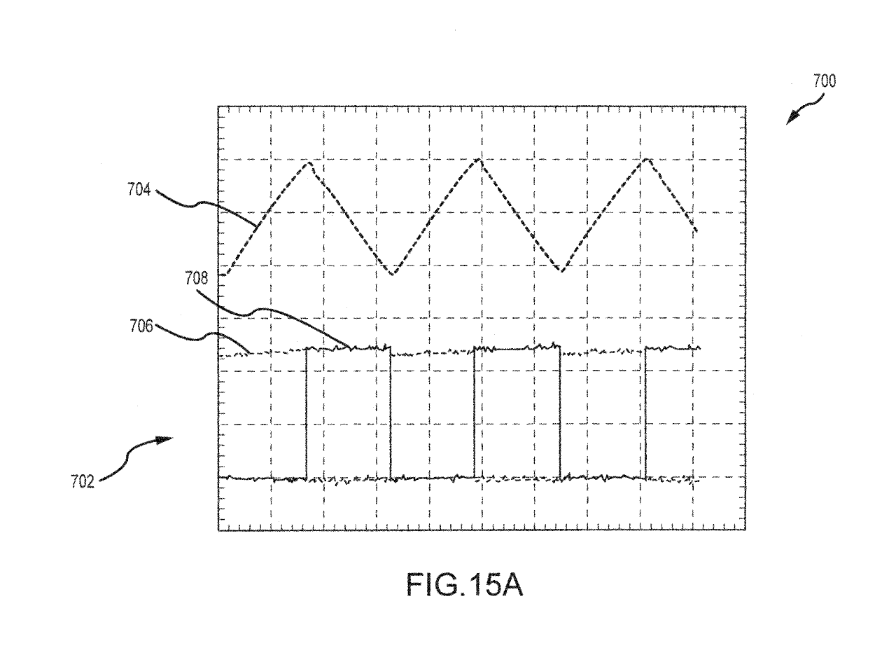

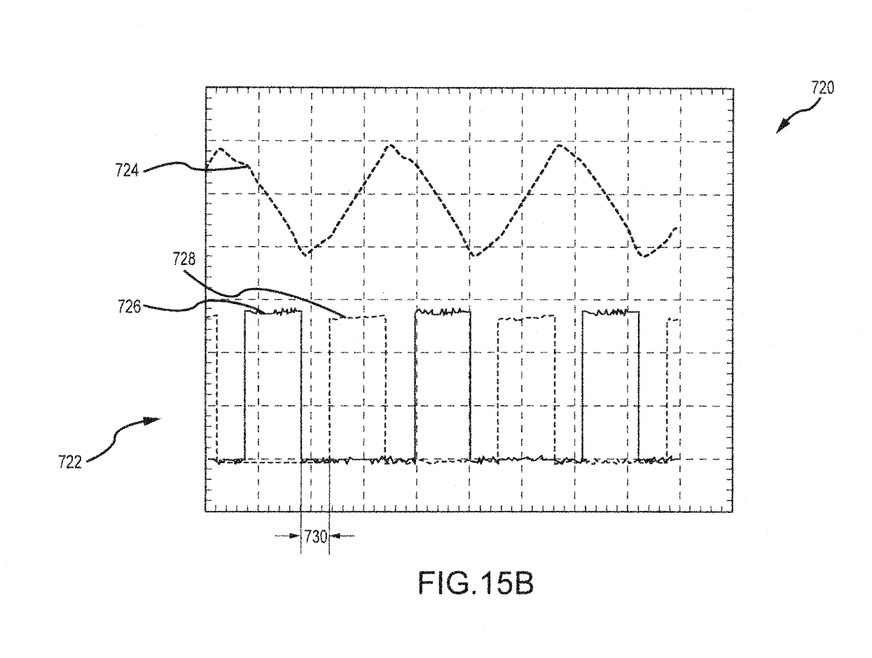

FIG. 15A is a graph 700 of a drive signal 702 used to drive shape memory members, such as shape memory members 312, 314 of actuator 300, to produce oscillating movement of a load such as load 320. The horizontal axis represents time and, for the drive signal 702, the vertical axis represents applied voltage. For example, a first drive signal portion 706 may drive shape memory member 312 and a second drive signal portion 708 may drive shape memory member 314. The corresponding position 704 of the load 320 is shown in the top half of the graph 700. For the position 704, the vertical axis represents angular position of the load 320. In the drive scheme illustrated by FIG. 15A, each shape memory member 312, 314 is sequentially driven in a non-overlapping fashion, i.e., substantially only one of the shape memory members 312, 314 is driven at a particular point in time and one of the shape memory members 312, 314 is substantially always being driven. This produces the motion pattern shown in the graph of the position 704 of the load 320 where the load 320 is substantially always being actively driven to one or the other of the ends points of its oscillating motion.

In actuator 300, when one of the shape memory members 312, 314 (the hot member) has been actuated such that it is at its substantially minimum operational length, the other shape memory member 312, 314 (the cool member) will be relatively cool and may contain a certain amount of elastic tension (e.g., spring load) due to elastic stretching. This does not unduly stress the hot member since it is a relatively small elastic tension. When the electrical current used to heat the hot member is removed, the cool member may reverse the direction of the load 320 due to the stored elastic energy within the cool member. Thus, it may not be necessary to always be driving one of the shape memory members 312, 314. Such a driving scheme 722 is illustrated in the graph 720 of FIG. 15B. In FIG. 15B, as in FIG. 15A, the horizontal axis represents time and, for the drive signal 722, the vertical axis represents applied voltage and for the position 724, the vertical axis represents angular position of the load 320. As shown, a time interval 730 between pulses 726, 728 may be incorporated. During the time interval, motion of the load 320 may be generated by the stored elastic energy to produce a motion profile 724 that is very similar to the profile 704 of FIG. 15A. Such a use of “rebound” (e.g., the expenditure of the stored elastic energy) may reduce overall power consumption of the actuator 300 as compared to the drive signal 702 of FIG. 15A. The elastically deformable members may also contribute to the rebound.

In an embodiment, the cool member may be heated such that it reaches its austenitic start temperature at the same time that the hot member cools to its martensitic start temperature. This procedure helps to prevent or limit the members from working directly against each other, which could cause excessive elastic tension and increase the risk of failure or reduced life of, in particular, the shape memory members. In this regard, the insulation level may be selected to produce the desired cooling rate that enables such balancing. Where the balancing is precisely controlled, the elastically deformable members may not be necessary.

The shape memory members 312, 314 may be configured such that prior to the application of energy to either shape memory members 312, 314, when they are both in a cooled (e.g., room temperature) state, the shape memory members 312, 314 may each be in elastic tension. This may enable the shape memory members 312, 314 to remain in contact with the cross shaft 332 prior to the application of energy to one of the shape memory members 312, 314. Furthermore, during operation, the shape memory members 312, 314 may be controlled such that each shape memory members 312, 314 is substantially always in some degree of elastic tension.

The drive signals used to drive the shape memory members 312, 314 may be capable of operating at relatively low voltages, such as, for example, voltages less than 35 V dc. Such low operating voltages may be beneficial in that they are within acceptable limits for devices to be inserted in patients. The actuator 300 may be operable to be driven at a frequency of 1 cycle per second or greater while meeting regulatory and/or other requirements for voltage levels and temperature (e.g., remaining below a maximum temperature while disposed within a patient).

### Examples

An actuator with first and second shape memory members capable of pivoting a load was constructed. The overall dimensions of the actuator were approximately 14 mm long with a diameter of 3 mm. The outer shell was made of stainless steel tubing and the end pieces were each made from alumina ceramic. The load was a piezoceramic 64 element ultrasound transducer array with a composite acoustic backing. The end pieces were center bored and defined the pivot axis for the load. The actuator was operated with a total angular range for the load of 44° (±22° from the home position) and had a maximum total angular range of 60°. The first and second shape memory members were in the form of 0.0015″ diameter Nitinol wire. The drive signal comprised a 10 Hz square wave of approximately 4.8 V dc. The actuator produced 10 Hz oscillating load movement producing a bidirectional scan rate for the ultrasound transducer array of 20 Hz. The 10 Hz oscillating load movement was limited by the hardware producing the 10 Hz square wave. In another exemplary dual shape memory member actuator, first and second shape memory members were in the form of 0.0015″ diameter Nitinol wire with parylene coating; immersed in water. The drive signal comprised a 6 Hz wave of approximately 4.5 V dc. The actuator produced 6 Hz oscillating load movement through an angular range of 50° (±25° from the home position) through 50,000 continuous, full sweeps. In another exemplary dual shape memory member actuator, a linearity of motion of a load of 10% was achieved using a triangular waveform and insulation on the first and second shape memory members. The insulation was 7 micron thick HSTF ePTFE polymer, and the actuator was run at 2.5 Hz at 1000× actual volume.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1. A catheter comprising:
an elongate catheter body;
a distal end portion supportably disposed at a distal end of said catheter body and defining an enclosed volume containing a fluid;
an ultrasound transducer immersed within said fluid and disposed for oscillating, pivotal movement through an angular range about a pivot axis within said enclosed volume, said pivot axis being fixed relative to said distal end portion; and,
first and second shape memory members operatively associated with said ultrasound transducer, wherein said first shape memory member can be actuated by causing a change in state of said first shape memory member, wherein said second shape memory member can be actuated by causing a change in state of said second shape memory member, wherein said first and second shape memory members are actuatable in at least partially-offset timed relation to affect at least a portion of said oscillating, pivotal movement of said ultrasound transducer.

2. A catheter as recited in claim 1, said first and second shape memory members being operatively associated with said ultrasound transducer within said enclosed volume, and further comprising:
first and second thermal insulation layers disposed about at least a portion of said first and second shape memory members, respectively, and immersed within said fluid.

3. A catheter as recited in claim 2, wherein said fluid is a liquid.

4. A catheter as recited in claim 3, wherein said first and second thermal insulation layers each comprise a fluoropolymer.

5. A catheter as recited in claim 4, wherein said first and second thermal insulation layers each comprise at least one material selected from a group consisting of:
polytetrafluoroethylene; and
expanded polytetrafluoroethylene.

6. A catheter as recited in claim 3, wherein said first and second thermal insulation layers each comprise at least one material that is hydrophobic.

7. A catheter as recited in claim 3, wherein said first and second thermal insulation layers each comprise at least one material that is microporous.

8. A catheter as recited in claim 3, wherein said first and second thermal insulation layers each have a thermal conductance of between about 0.05 W/mK and 0.08 W/mK when measured at about 25° C.

9. A catheter as recited in claim 8, wherein said first and second thermal insulation layers comprise expanded polytetrafluoroethylene.

10. A catheter as recited in claim 3, further comprising:
first and second outer layers adherently disposed about said first and second thermal insulation layers, respectively, and immersed within said fluid.

11. A catheter as recited in claim 10, said first and second outer layers each having a dielectric withstand voltage of at least about 500 kV/m.

12. A catheter as recited in claim 10, wherein said first and second outer layers each comprise a hydrophobic material.

13. A catheter as recited in claim 12, wherein said first and second outer layers have a surface energy of less than about 50 dyn/cm².

14. A catheter as recited in claim 1, wherein said first shape memory member is actuatable to rotate said ultrasound transducer in a first direction about said pivot axis, and wherein said second shape memory member is actuatable to rotate said ultrasound transducer in a second direction about said pivot axis, said first direction being opposite to said second direction.

15. A catheter as recited in claim 14, wherein said first and second shape memory members are defined by corresponding first and second shape memory wire lengths, respectively, wherein a first end of said first shape memory wire length is interconnected in fixed relation to one of said distal end portion and said ultrasound transducer on one side of said pivot axis, and wherein a first end of said second shape memory wire length is interconnected in fixed relation to one of said distal end portion and said ultrasound transducer on another side of said pivot axis opposite to said one side.

16. A catheter as recited in claim 15, wherein said first shape memory wire length is interconnected to a corresponding other one of said ultrasound transducer and said distal end portion at a first interconnection location, and wherein said second shape memory wire length is interconnected to a corresponding other one of said ultrasound transducer and said distal end portion at a second interconnection location, said first and second locations being on opposite sides of said pivot axis.

17. A catheter as recited in claim 16, wherein each of said first and second shape memory wire lengths having corresponding second ends interconnected in fixed relation to said corresponding one of said distal end portion and said ultrasound transducer, and wherein said first and second shape memory wire lengths are interconnected between their corresponding first and second ends to the corresponding other one of the distal end portion and the ultrasound transducer at said corresponding first and second interconnection locations, respectively, said first and second interconnection locations being offset on opposite sides of said pivot axis.

18. A catheter as recited in claim 17, wherein said first and second shape memory wire lengths each include corresponding first and second portions that define corresponding first and second included angles therebetween, respectively.

19. A catheter as recited in claim 18, wherein said first and second shape memory wire lengths are arranged so that said first and second included angles increase and decrease in response to corresponding actuation and de-actuation of said first and second shape memory wire lengths, respectively.

20. A catheter as recited in claim 16, wherein said first and second interconnection locations are substantially equidistance from said pivot axis of said ultrasound transducer.

21. A catheter as recited in claim 20, wherein said first and second shape memory wire lengths are symmetrically disposed relative to said ultrasound transducer.

22. A catheter as recited in claim 16, wherein said first interconnection location is located on said another side of said pivot axis, and wherein said second interconnection location is located on said one side of said pivot axis.

23. A catheter as recited in claim 15, wherein said first shape memory wire length has a corresponding second end interconnected in fixed relation to said corresponding one of said distal end portion and said ultrasound transducer, and further comprising:
a engagement member interconnected in fixed relation to the other one of said distal end portion and said ultrasound transducer to which said first and second ends of first shape memory wire length are interconnected, wherein said first shape memory wire length engages in said engagement member to rotate said ultrasound transducer in said first direction during actuation of said first shape memory wire length.

24. A catheter as recited in claim 23, wherein said second shape memory wire length has a corresponding second end interconnected in fixed relation to said corresponding one of said distal end portion and said ultrasound transducer, wherein said second shape memory wire length engages in said engagement member to rotate said ultrasound transducer in said second direction during actuation of said second shape memory wire length.

25. A catheter as recited in claim 15, wherein the first and second shape memory wire lengths may comprise physically-separate first and second wires, respectively.

26. A catheter as recited in claim 15, wherein the first and second shape memory wire lengths are defined by corresponding different segments of a continuous shape memory wire.

27. A catheter as recited in claim 1, further comprising:
a drive energy source for repeatedly providing first and second energy signals during corresponding first and second time periods to said first and second shape memory members to change the state of said first and second shape memory members, respectively, with a first time interval between an end of each first time period and a start of each second time period, wherein at least said second shape memory member is provided to be in elastic tension during at least a portion of each first time interval so that the second shape memory member is operable to affect at least a portion of said oscillating, pivotal movement of said ultrasound transducer during each first time interval.

28. A catheter as recited in claim 27, wherein said drive energy source repeatedly provides said first and second energy signals with a second time interval between an end of each second time period and start of each first time period, and wherein said first shape memory member is provided to be in elastic tension during at least a portion of each second time interval so that the first shape memory member is operable to affect at least a portion of said oscillating, pivotal movement of said ultrasound transducer during each second time interval.

29. A catheter as recited in claim 28, wherein said first and second shape memory members are provided to affect different portions of said oscillating, pivotal movement of the ultrasound transducer corresponding with opposite end portions of said angular range.

30. A catheter as recited in claim 1, further comprising:
a first magnetic member supportably connected to one of said distal end portion, and said ultrasound transducer, and located to affect at least a first portion of said oscillating, pivotal movement of said ultrasound transducer.

31. A catheter as recited in claim 30, wherein said first magnetic member comprises a permanent magnet.

32. A catheter as recited in claim 30, wherein said first magnetic member comprises an electromagnetic member.

33. A catheter as recited in claim 30, further comprising:
a second magnetic member supportably connected to one of said distal end portion and said ultrasound transducer, and located to affect at least a second portion of said oscillating, pivotal movement of said ultrasound transducer.

34. A catheter as recited in claim 33, wherein said first and second portions of said oscillating, pivotal movement of the ultrasound transducer correspond with opposite end portions of said predetermined angular range.

35. A catheter as recited in claim 33, wherein each of said first and second magnetic members is operable to apply one of an attractive force and a repulsive force to at least one magnetizable member interconnected to the corresponding other one of said distal end portion and said ultrasound transducer.

36. A catheter as recited in claim 1, wherein said distal end portion is selectively positionable across a range of angles relative to the catheter body.

37. A catheter as recited in claim 1, wherein said distal end portion is selectively rotatable relative to the catheter body.

38. A method for use in a catheter having an ultrasound transducer immersed within a fluid and disposed for pivotal movement about a pivot axis within an enclosed volume defined by a distal end portion supportably disposed at a distal end of an elongate catheter body, comprising:
first actuating a first shape memory member operatively associated with said ultrasound transducer to pivot said ultrasound transducer in a first direction;
second actuating a second shape memory member operatively associated with said ultrasound transducer to pivot said ultrasound transducer in a second direction opposite to said first direction;
repeating said first and second actuating steps in accordance with a predetermined cycle to affect oscillating, pivotal movement of said ultrasound transducer through an angular range relative to the pivot axis; and,
operating said ultrasound transducer to at least one of transmit and receive acoustic signals through said fluid during at least a portion of each occurrence of at least one of said first and second actuating steps.

39. A method as recited in claim 38, wherein said first actuating step comprises:
first applying a first electrical signal to said first shape memory member to change said first shape memory member from a first configuration to a second configuration and thereby impart a first force to said ultrasound transducer; and,
wherein said second actuating step comprises:
second applying a second electrical signal to said second shape memory member to change said second shape memory member from a first configuration to a second configuration and thereby impart a second force to said ultrasound transducer.

40. A method as recited in claim 39, said first and second shape memory members being defined by corresponding first and second shape memory wire lengths, respectively, wherein said first shape memory wire length shortens during said first applying step, and said second shape memory wire length shortens during said second applying step.

41. A method as recited in claim 40, wherein each of said first and second shape memory wire lengths have corresponding first and second ends interconnected in fixed relation to said distal end portion, and wherein said first and second shape memory wire lengths are interconnected between their corresponding first and second ends to said ultrasound transducer at corresponding first and second interconnection locations offset from said pivot axis, said first and second interconnection locations being on opposite sides of said pivot axis.

42. A method as recited in claim 41, wherein said first and second shape memory wire lengths each include corresponding first and second portions that extend away from their corresponding first interconnection location and second interconnection location to define corresponding first and second included angles, respectively, and wherein the method further comprises:
increasing said first included angle and decreasing said second included angle during said first applying step; and,
increasing said second included angle and decreasing said first included angle during said second applying step.

43. A method as recited in claim 39, further comprising:
using said first force to return said second shape memory member from its second configuration to its first configuration; and,
using said second force to return said first shape memory member from its second configuration to its first configuration.

44. A method as recited in claim 43, wherein said oscillating, pivotal movement of said ultrasound transducer occurs at a rate between 1 and 50 Hz.

45. A method as recited in claim 43, wherein said oscillating, pivotal movement of said ultrasound transducer occurs at a rate between 8 and 30 Hz.

46. A method as recited in claim 43, wherein said oscillating, pivotal movement of said ultrasound transducer occurs at a rate of at least 10 Hz.

47. A method as recited in claim 43, wherein said oscillating, pivotal movement of said ultrasound transducer occurs at a rate of at least 50 Hz.

48. A method as recited in claim 39, wherein said predetermined cycle includes a first time interval between an end of the first applying step and a start of the second applying step, and wherein said method further comprises:
employing an elastic response of said second shape memory member during each first interval to initiate pivotal movement of said ultrasound transducer in said second direction.

49. A method as recited in claim 48, wherein said predetermined cycle includes a second time interval between an end of the second applying step and a start of the first applying step, and wherein said method further comprises:
employing an elastic response of said first shape memory member during each second interval to initiate pivotal movement of said ultrasound transducer in said first direction.

50. A method as recited in claim 39, wherein at least one magnet is interconnected to one of said distal end portion and said ultrasound transducer, and wherein the method further comprises:
employing said at least one magnet to apply a magnetic force to said ultrasound transducer to affect at least a portion of said oscillating, pivotal movement.

51. A method as recited in claim 39, wherein a first magnet is interconnected to said ultrasound transducer and a second magnet is interconnected to said distal end portion, and wherein the method further comprises:
employing said first magnet and said second magnet to apply magnetic forces to affect different portions of said oscillating, pivotal movement.

52. A method as recited in claim 38, further comprising:
operating said ultrasound transducer to receive acoustic signals through said fluid during at least a portion of each occurrence of at least one of said first and second actuating steps and to provide a corresponding output signal; and,
using said output signal in an ultrasound imaging system.

53. A method as recited in claim 38, further comprising:
operating said ultrasound transducer to receive acoustic signals through said fluid during at least a portion of each occurrence of at least one of said first and second actuating steps and to provide a corresponding output signal; and,
processing said output signal utilizing a computer processor to generate at least three-dimensional images.

54. A method as recited in claim 53, further comprising:
displaying said three-dimensional images at a user interface.

55.-88. (canceled)
